(19) United States
(12) Patent Application Publication
(10) Pub. No.: US 2025/0085274 A1
(43) Pub. Date: Mar. 13, 2025

(54) BREVETOXIN DETECTION DEVICE, SYSTEM, AND METHOD

(71) Applicant: Mote Marine Laboratory, Inc., Sarasota, FL (US)

(72) Inventors: Dana L. Wetzel, Sarasota, FL (US); Tracy A. Sherwood, Sarasota, FL (US)

(21) Appl. No.: 18/823,598

(22) Filed: Sep. 3, 2024

Related U.S. Application Data
(60) Provisional application No. 63/535,889, filed on Aug. 31, 2023.

(51) Int. Cl. G01N 33/53; G01N 33/569

(52) U.S. Cl. CPC G01N 33/5308; G01N 33/56961; G01N 2469/10

(57) ABSTRACT

A method to detect the presence of parent brevetoxin (BT) and brevetoxin metabolites (BTXs) in shellfish and aquatic samples is provided. The method may include contacting a sample with a binding molecule comprising an aptamer, and determining if the binding molecule binds a Brevetoxin antigen in the sample.

### CROSS-REFERENCE TO RELATED APPLICATIONS

This application claims priority to U.S. Provisional Application No. 63/535,889, filed on Aug. 31, 2023, the disclosure of which is incorporated herein by reference.

### TECHNICAL FIELD

The subject matter disclosed herein is generally directed to the detection of toxins, and more specifically, methods, devices, and systems to detect a presence of brevetoxins, including parent brevetoxins (BT) and brevetoxin metabolites (BTX) in, for example, shellfish and aquatic samples.

### BACKGROUND

Red tide algal blooms are often caused by Karenia brevis, an algal species that produces several brevetoxin (BT) compounds, including both parent BT compounds and metabolite BT species (collectively, BTXs). Although most prevalent along the southwest Florida coast, and sometimes lasting over a year, red tide blooms have occurred along the entire US and Mexico Gulf coasts, and along the Atlantic coast as far north as North Carolina. BTXs are a neurotoxic to a wide variety of organisms. For example, in some instances, human consumption of bivalve mollusks (e.g., clams, oysters, mussels and scallops) containing sufficiently high BTX levels can lead to neurotoxic shellfish poisoning (NSP). Though BTXs tend to accumulate most significantly in shellfish, contamination of other marine organisms also commonly occurs.

Bivalve shellfish production represents a large and growing segment of the US and global seafood industry attempting to keep pace with seafood demand and a growing human population. Shellfish aquaculture (e.g., clams, oysters, scallops, mussels, crabs, lobster, squid, and snails) holds a place of significant economic and ecological importance. Commercial industries for shellfish, especially within Florida, are considerable. Florida has long benefited from shellfish farming, with a state economic impact estimated at over $37 million. In just over three decades, hard clams represented the single most important aquaculture food item produced by Florida's aquatic growers. In 2019, North Carolina and Texas enacted laws to further enhance and support current aquaculture industries (NC) and establish a new shellfish industry (TX). Southwest Florida produces very desirable white and salty shellfish due in part to the greater concentration of white sand compared to darker sediment found in other areas of the state. These ideal environmental conditions also allow for the cultivation of various new species, such as the highly sought sunray venus clam, thus, further diversifying the industry and expanding Florida's growth potential.

However, the increasing frequency and duration of Gulf of Mexico farm closures due to toxic red tide blooms have severely damaged southwest Florida's shellfish industry and reduced the availability of safe shellfish for consumers. Shellfish farmers in affected areas have seen an escalating number of red tide-related farm closures, resulting in lost revenue, jobs, and economic uncertainty valued in millions of dollars. Socio-economic hardships of red tide blooms cripple shellfish farmers and workers in processing, distribution, and retailing, making the US less competitive on a domestic and global scale and limiting product availability.

The highly stochastic nature of red tide blooms can create considerable uncertainty for farmers and regulators tasked with supervising the reopening of shellfish harvest areas (SHAs). The complex regulatory shellfish seafood safety procedures require farmers to leave a potentially safe product in the water or hold the harvested product for extended periods until its status can be cleared using the current, time-consuming laboratory analyses. This conundrum often results in a lag time where farmers must harvest and hope for a good outcome. In both instances, farms can suffer significant economic losses affecting cash flow and may cause the product to size out of the market, thus slashing the crop value. Harvesting product with uncertain status creates the risk that large amounts of the product must be destroyed or returned to the farm (with high mortality), leading to even more economic loss. The longer a shellfish harvest area is closed, the more revenue is lost and may not be recovered.

The National Shellfish Sanitation Program (NSSP) is the Federal/State cooperative program recognized by the US Food and Drug Administration (FDA) and the Interstate Shellfish Sanitation Conference (ISSC) for the sanitary control of shellfish produced and sold for human consumption. In Florida, the Division of Aquaculture within the Florida Department of Agriculture and Consumer Services (FDACS) oversees, monitors, and regulates NSSP guidelines for harvesting shellfish during a red tide. In the US, the shellfish assessment of NSP toxins has been driven by the regulatory framework designed to protect public health.

There are two conventional protocols that the NSSP has approved for the assessment of NSP toxins in biological samples: a) the mouse bioassay (MBA) and b) an enzyme-linked immunoassay (ELISA).

The MBA involves testing live animals, requiring a research facility that is approved, certified, and inspected by the USDA, making this a costly method to sustain. It is a slow and labor-intensive method resulting in a lack of time-critical information to approve the reopening of shellfish harvest areas. The test does not measure toxin levels as it is not calibrated with known concentrations of BTXs. Instead, the test is a non-specific measure of the relative toxicity of a shellfish extract which does not determine the specific toxins responsible for the observed effects (mortality). Therefore, the results solely report the number of units necessary to cause mortality in some mice, not conclusive evidence of BTX toxicity. This deficiency raises the possibility of false positives, something the shellfish farming community believes happens with some frequency. Further, MBA sample throughput is limited (often to two samples/week) by the availability of suitable mice. This limitation often leaves growers unable to have their product tested, compounding the economic losses.

Currently, only ELISA or MARBIONC ELISA is approved as a limited-use method for assessing BTXs in oysters, hard clams, and sunray venus clams. ELISA is an anti-BTX polyclonal Ab (pAb) assay specific only to B-type BTXs, which greatly reduces the number of false positives that occur with the MBA. NSSP guidelines state that when ELISA results are ≤1.6 ppm (clams) or ≤1.8 ppm (oysters), the ELISA may substitute for the MBA for purposes of controlled relaying, controlled harvest end-product testing or to reopen a previously closed area. Concentrations of BTXs measured above those thresholds using this ELISA require additional MBA testing. While an improvement over the MBA in efficiency, the current approved ELISA also has limitations. The Florida Division of Aquaculture monitors over 1,200 water quality stations in 38 SHAs, encompassing over 1.3 million acres of coastal waters. During mass closures of SHAs due to red tide, timely analysis of shellfish from all impacted areas may be limited due to available resources, including staff. From the extraction of samples to data generation, a skilled technician can theoretically complete up to 20 analyses daily with the ELISA. However, sample re-runs due to commonly encountered concentration/dilution minima and maxima range exceedances that violate the quality assurance and quality control requirements can reduce the number of samples that can be completed. Animals are required to produce pAbs used in the assay, which results in high batch variability. Moreover, the ELISA does not detect the presence of A-type backbone BTXs, the B-type backbone BTX-B4, or any open A-ring toxins.

In order to thrive, the industry needs innovative technology for red tide toxin detection that can have a correlation with seafood safety regulatory protocols. From the growers' perspective, current procedures can appear cumbersome, confusing, expensive, and, most importantly, slow.

### SUMMARY OF SOME EXAMPLE EMBODIMENTS

According to some example embodiments, a binding molecule for use in detection of brevetoxins in a sample is described. The binding molecule may comprise an aptamer configured to bind to a brevetoxin antigen and a detection component having a detection property. In this regard, the detection component may be operably coupled to the aptamer and configured to facilitate detection of the brevetoxin in the sample, via the detection property, upon binding of aptamer to the brevetoxin antigen.

According to some example embodiments, a method of detecting a brevetoxin in a sample is described. The method may comprise applying a detector solution to the sample to cause an aptamer of a binding molecule of the detector solution to bind with a targeted brevetoxin antigen. Upon binding of the aptamer to the brevetoxin antigen, a detection property of the detection component of the aptamer is activated. The method may further comprise detecting, via a sensor, a response signal from the sample based on the detection property. Further, the method may comprise determining, by control circuitry, a presence of the brevetoxin in the sample based on the response signal from the sample.

According to some example embodiments, a test kit for detecting a brevetoxin in a sample is described, the test kit may comprise a holder configured to receive the sample and a detector solution comprising a binding molecule. The binding molecule may comprise an aptamer configured to bind to a brevetoxin antigen within the sample, and a detection component having a detection property. The detection component may be operably coupled to the aptamer and configured to facilitate detection of the brevetoxin in the sample, via the detection property, upon binding of aptamer to the brevetoxin antigen.

Some example embodiments, including those described above, also provide devices, systems, and methods for detecting both A- and B-type BTXs (brevetoxin compounds including both parent BT compounds and metabolite BT species) in samples, such as shellfish and aquatic samples, using a BTX-binding aptamer. The BTX-binding aptamer may include nucleic acid sequences which can recognize and bind a target of interest (ligand), such as an antigen of a brevetoxin. In one aspect, binding molecules may bind with both A- and B-type BTXs via the BTX-binding aptamer.

According to some example embodiments, devices, systems, and methods using such BTX-binding aptamers may be used to detect BTXs from a variety of sample sources. Such sample sources may include shellfish, or other aquatic samples, taken from any naturally occurring or manmade water source or site where the shellfish are reared, housed, or wild-caught, and may, for example, represent water samples taken from an aquaculture water source; water samples taken from an aquaculture facility (e.g., a pond, pen, tank, cage or other fish-rearing enclosure); samples taken from aquaculture effluent; samples taken from fishing vessels (e.g., water samples taken from holding tanks or from seawater); or samples taken from fish (e.g., as fish mucus, fish gill swabs, skin swabs, fish tissue, fish tissue extracts or fish mucus extracts).

According to some example embodiments, a binding molecule may comprise an aptamer that is cross-reactive with one or more brevetoxin antigens. According to some example embodiments, the aptamer may comprise a sequence having 60% or greater, 65% or greater, 70% or greater, 75% or greater, 80% or greater, 85% or greater, 90% or greater, 95% or greater, 97% or greater, 98% or greater, 99% or greater, or 100% identity to any one of SEQ ID NOs: 1 to 50 or SEQ ID Nos: 1 to 20 (illustrated below as BTXAP 1 to 50 and BTXAP 1 to 20).

In another aspect, the invention provides a method for detecting a BTX antigen. The method includes contacting a sample with a binding molecule described herein, and then determining if the binding molecule binds a BTX antigen present in the sample.

In another example, the invention provides a method for creating or generating aptamers that bind to a BTX antigen. The method includes the step of providing a BTX antigen immobilized on a support, contacting an oligonucleotide library with the support; and then identifying oligonucleotides from the library that specifically bind to the BTX antigen. Oligonucleotides that are BTX antigen-binding aptamers are identified while undesirable targets are eliminated.

In another example, the binding molecules may be used as part of an assay to detect BTX, e.g., a rapid fluorescent aptamer assay (FAA) or enzyme-linked aptasorbent assay (ELASA).

Such devices, systems, and methods may allow for broadened sensitivity, increased sample throughput, lower cost, and minimal analytical expertise for a more rapid turnaround. This may significantly improve the outlook for shellfish farmers and end users alike.

While various BTX detection prototypes are discussed, it is contemplated that additional or different applications may be developed as well using high affinity Brevetoxin aptamers. The aforementioned prototypes are merely illustrative.

### BRIEF DESCRIPTION OF THE DRAWINGS

An understanding of the features and advantages of the present disclosure will be obtained by reference to the following detailed description that sets forth illustrative embodiments, in which the principles of the disclosure may be utilized, and the accompanying drawings of which:

FIG. 1A depicts the organic compound structure of brevetoxin A-type backbone, according to some example embodiments.

FIG. 1B depicts the organic compound structure of brevetoxin B-type backbone, according to some example embodiments.

FIG. 2A depicts a fluorescent aptamer assay (FAA), according to some example embodiments.

FIG. 2B depicts one embodiment of an enzyme-linked aptasorbent assay (ELASA), according to some example embodiments.

FIG. 2C depicts another embodiment of an enzyme-linked aptasorbent assay (ELASA), according to some example embodiments.

FIG. 3 depicts preparing a single-stranded library for selection during systematic evolution of ligands by exponential enrichment (SELEX), according to some example embodiments.

FIG. 4 depicts the performance of a counter selection during SELEX, according to some example embodiments.

FIG. 5 depicts positive selection during SELEX, according to some example embodiments.

FIG. 6 depicts single-stranded library regeneration during SELEX, according to some example embodiments.

FIG. 7 depicts fluorescent results over successive BTX selection cycles, according to some example embodiments.

FIG. 8 depicts a ratio of positive to negative fluorescence at each BTX selection cycle, according to some example embodiments.

FIG. 9A depicts a normalized fluorescence calibration curve for BTX-7, according to some example embodiments.

FIG. 9B depicts a comparison of an early aptamer's sensitivity to BTX-2 with five, second-generation aptamers' sensitivity to BTX-2, according to some example embodiments.

FIG. 10 depicts the detection of BTX-7 with a dynamic concentration range from 0.02 ppm to 3.0 ppm by indirect competitive enzyme-linked aptasorbent assay (ELASA), according to some example embodiments.

FIG. 11 depicts the regression coefficients for the percent inhibition of the BTX-3 with the MARBIONC ELISA and BTX-7 with the ELASA over the same concentration range, according to some example embodiments.

FIG. 12 depicts a flowchart of an example method for detecting a brevetoxin, according to some example embodiments.

The figures herein are for illustrative purposes only and are not necessarily drawn to scale.

### DETAILED DESCRIPTION

Some example embodiments will now be described more fully with reference to the accompanying drawings, in which some, but not all example embodiments are shown. Indeed, the examples described and pictured herein should not be construed as being limiting as to the scope, applicability, or configuration of the present disclosure. Rather, these example embodiments are provided so that this disclosure will satisfy applicable legal requirements. Like reference numerals refer to like elements throughout. The term “or” as used herein is defined as the logical or that is true if either or both are true.

In view of the foregoing, according to some example embodiments, a BTX-binding molecule comprising a BTX-binding aptamer may be implemented that is cross-reactive with brevetoxins (BTXs) to facilitate the detection of such toxins in water samples or other samples, for example, taken from shellfish or the like. A brevetoxin may be any of several neurotoxic substances that are produced by a dinoflagellate, such as Karenia brevis, that may be found in red tides and may cause massive fish die-offs and sicken or kill marine mammals and birds, and may cause humans to experience respiratory symptoms (such as coughing or shortness of breath) when inhaled and experience neurotoxic shellfish poisoning (NSP) when ingested. Moreover, brevetoxins are a suite of lipid-soluble cyclic polyether compounds comprising almost 90 parent, analog, and metabolite members responsible for NSP.

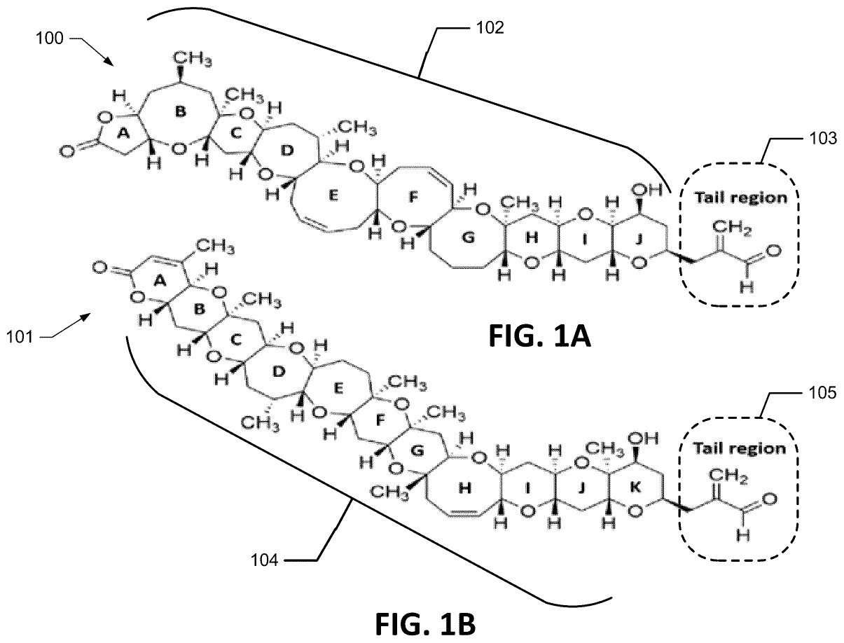

As shown in FIG. 1A and FIG. 1B, BTXs may have different and distinct structures. FIG. 1A illustrates a BTX 100 having an A-type backbone and FIG. 1B illustrates a BTX 101 having a B-type backbone. Both may be characterized structurally as relatively linear with a bend mid-molecule. A BTX may therefore comprise a backbone and a side chain referred to as a tail region. The backbone may be a central chain of covalently bonded atoms (typically Carbon) that make up a primary structure of a molecule. The tail region may be defined as a side chain that is attached to an end of the backbone.

In this regard, BTX 100, in FIG. 1A, comprises a backbone 102 and a tail region 103. The tail region 103 may be defined as a side chain that is attached to an end of the backbone 102. The backbone 102 may be an A-type backbone having a suite of cyclic polyethers (illustrated as extending from A to J, with A being at one end and J being at the other end). Cyclic polyethers A and J of the backbone 102 may be the ends of the backbone 102 due to only being bonded to one other cyclic polyether. The tail region 103 may comprise an unsaturated aldehyde, which may be bonded to the cyclic polyether J.

Additionally, BTX 101, in FIG. 1B, comprises a backbone 104 and a tail region 105. The tail region 105 may be defined as a side chain that is attached to an end of the backbone 104. The backbone 104 may be a B-type backbone having a suite of cyclic polyethers (illustrated as extending from A to K, with A being at one end and K being at the other end). Cyclic polyethers A and K of the backbone 104 may be the ends of the backbone 102 due to only being bonded to one other cyclic polyether. The tail region 105 may comprise an unsaturated aldehyde, which may be bonded to the cyclic polyether K. While the backbones of BTX 100 and BTX 101 may be different, the tail regions 103 and 105 may be the same. In this regard, both the BTX 100 and the BTX 101 may have respective tail regions comprising, for example, identically-structured unsaturated aldehydes.

Some of the most common BTX compounds are provided in Table 1:

TABLE 1

| A Toxins | CR* | B Toxins | CR | B Toxins | CR |
|---|---|---|---|---|---|
| BTX-1 | N | BTX-2 | Y | BTX-B3 | — |
| BTX-7 | — | BTX-3 | Y | BTX-B4 | N |
| BTX-10 | — | BTX-5 | Y | BTX-B5 | Y |
| BTX-A | — | BTX-6 | N | S-deoxyBTX-B2 | — |
| BTX-A1 | — | BTX-8 | — | cysteinylglycine-PbTx-B | — |
| BTX-A2 | — | BTX-9 | Y | g-glytamylcysteine-PbTx-B | — |
| S-deoxyBTX-A1 | — | BTX-B | — | N-hexadecanoyl-cysteine- | — |
| | | | | PbTx-B | |
| S-deoxyBTX-A2 | — | BTX-B1 | — | N-hexadecanoyl-cysteine- | — |
| | | | | PbTx-B-SO | |
| BTX-A5 | — | BTX-B2 | Y | N-tetradecanoylcysteine- | — |
| | | | | cysteine-PbTx-B | |
| | | S-deoxyBTX-B2 | — | N-tetradecanoylcysteine- | — |
| | | | | cysteine-PbTx-B-SO | |

*CR = cross-reactive with ELISA as evaluated using a polyclonal antibody ELISA assay.
— = Not evaluated with ELISA.

Parent compounds BTX-1 (an A-type brevetoxin) and BTX-2 (a B-type brevetoxin) are considered the most toxic of all BTXs. BTX-1 and BTX-2 have different backbones and share a common unsaturated aldehyde tail region. BTX-3 is the most prevalent BTX found in shellfish and is a reduced form of BTX-2, sharing a common B-type backbone but with a different tail region. Metabolites of the parent BTXs in shellfish also contribute as causative agents for NSP. As such, BTX metabolites testing and monitoring of shellfish beds may also include techniques for testing for such metabolites to obtain increasingly accurate and complete assessments of the toxicity of a shellfish. As such, in accordance with some example embodiments, methods, devices, and systems are provided herein to detect, for example, a presence of parent BTX and BTX metabolites in shellfish and aquatic samples.

According to some example embodiments, a BTX aptamer identification approach may be implemented to determine BTX-binding aptamers for use in brevetoxin detection. As used herein, an “aptamer” may be an oligonucleotide (a polymer of nucleic acid residues) that adopts a configuration (e.g., a tertiary configuration) that folds into a stable complex capable of binding to an antigen or many antigens. Moreover, an aptamer may be oligomer of artificial single-stranded deoxyribonucleic acid (ssDNA), ribonucleic acid (RNA), xeno nucleic acid (XNA), or a peptide that binds to a specific target molecule or family of molecules. According to some example embodiments, in addition to having, for example, a nucleic acid sequence, an aptamer may be folded into a shape that may facilitate binding. Additionally, as used herein, an aptamer-based “binding molecule” may refer to a compound that includes an aptamer per se. Further, in some instances, a binding molecule may also include other components such as one or other non-nucleic acid/nucleotide moieties, such as detection moieties, including but not limited to, fluorophores, drugs, or other non-nucleic acid polymers. Accordingly, a BTX-binding aptamer may be an aptamer that has been determined to bind with a BTX.

Relative to the use of antibodies, in some instances, aptamers can have tremendous advantages as a detection tool. For example, in some instances, aptamers may be inexpensive, may be easy to synthesize, may eliminate batch-to-batch variations (which can be a common problem with polyclonal antibodies), may have higher sensitivity and specificity, may be more thermally stable, may maintain structural configurations over repeated cycles of denaturation/renaturation, and can be used in various aptasensor formats, such as lateral flow, colorimetric, and fluorescent. Alternatively, antibody production can, in some instances, require in-vivo synthesis, require heightened temperatures, have high pH sensitivity considerations, have time-limited usability due to limited shelf-life and stability, and have considerations for high batch variability and expense.

One valuable property of aptamers is the ease with which aptamers can be chemically modified and engineered to generate aptamer-conjugates. According to some example embodiments, aptamer conjugates can increase the interaction capabilities of aptamers with their targets, mitigate background noise, and have other benefits.

While aptamers for brevetoxin detection may be determined in a variety of ways, according to some example embodiments, a aptamer may be identified and produced through, for example, a systematic evolution of ligands by exponential enrichment (SELEX) process, a selected and amplified binding site (SAAB) process, or a cyclic amplification and selection of targets (CASTing) process. For example, SELEX can produce oligonucleotides that can be either single-stranded DNA or RNA molecules with specific binding properties to one or more target ligands (e.g., antigens). A SELEX process may isolate ligand-binding oligonucleotides from large libraries of random synthetic oligonucleotides to determine strong binding aptamers to the desired ligand. In one example, SELEX may be used to yield specific acid sequences from a starting pool of sequences through repeated selection cycles and polymerase chain reaction (PCR) amplification.

Thus, according to some example embodiments, methods for isolating an aptamer that specifically binds to one or more BTX antigen(s) have be employed to determine an aptamer that may be implemented in the context of a brevetoxin detector or the like. In regard, according to some example embodiments, a BTX may comprise one or more antigens that may be leveraged for binding with, for example, an aptamer and the BTX antigen-to-aptamer binding may be leveraged for detection of the presence of the BTX. As mentioned above, such aptamer may be referred to as a BTX-binding aptamer. Due to the binding, subsequent detection may be performed based on the bound aptamer to the selected BTX antigen to detect the presence and possibly a relative amount of the BTX. Example BTX antigens that may be targeted for binding with a BTX-binding aptamer may include those provided in Table 1 or any other BTXs with A- or B-type backbones.

In operation, according to some example embodiments, a BTX antigen or mixture of BTX antigens may be immobilized on a support (e.g., a solid support). An aptamer library (e.g., an oligonucleotide library) with a plurality of aptamers (or oligonucleotides) may be applied to contact the BTX antigen(s), and the aptamers that bind the BTX antigens may be isolated and separated from those that do not. In some example embodiments, the SELEX method can further include eluting or extracting one or more aptamer from the BTX antigen that has been immobilized on a support based on a stringency threshold. In this regard, the eluted BTX-binding aptamer may be one that has a high affinity for a BTX antigen (e.g., having a dissociation constant in the range of pico molar units (pM) to micro molar units (μM)).

After the eluted BTX-binding aptamer or aptamers have been isolated, a population of such BTX-binding aptamers can be amplified by a process, such as, for example, a polymerase chain reaction (PCR). In this regard, an aptamer or oligonucleotide library may be used where the aptamers or oligonucleotides have, for example, a standardized 5′ end with a specific nucleotide sequence or a standardized 3′ end with a specific nucleotide sequence. Further, primers complementary to 5′ or 3′ ends may be used in a PCR reaction to amplify the eluted and isolated BTX-binding aptamer or aptamers.

The process, for example, of contact exposure of a library of aptamers and then eluting, isolating, and amplifying, as described above, may be repeated any number of times with the same or different libraries. When repeated using the same library or a selected subset due to elimination of non-binding aptamers, the eluted BTX-binding aptamers may be applied to the support with an immobilized BTX antigen, allowing the BTX-binding aptamers to bind to the support and then the antigens may be eluted again, under conditions of increasing stringency (e.g., those having a lower dissociation constant of the given BTX antigen). As such, according to some example embodiments, an aptamer may be distinguished from an aptamer or oligonucleotide library as a nucleic acid species that may be determined or engineered through repeated selection rounds (e.g., in vitro selection) to bind to a BTX antigen, and, in some example embodiments, increasing stringency. According to some example embodiments, in some practice modes, the amplified oligonucleotides' primer regions may be removed after the desired aptamers are isolated as part of the process.

From the selection process, a BTX-binding aptamer may be isolated that has a nucleic acid sequence with one or more unpaired nucleic acid bases and one or more paired nucleic acid bases (such as in the form of base-paired stems) when the aptamer is folded into a double-stranded configuration. One or more unpaired nucleic acid bases may form a binding pocket that can bind to the BTX antigen.

According to some example embodiments, BTX-binding aptamers may have one or a plurality of stems. For example, such candidate aptamers can have a number of stems in the range of 1 to 6, in the range of 1 to 5, in the range of 1 to 4, in the range of 1 to 3, or 2, or 1. In those one or more stems of the aptamer there can be a number of base pairs which form the stem, the number of base pairs being in the range of 1 to 10, in the range of 1 to 9, in the range of 1 to 8, in the range of 1 to 7, in the range of 1 to 6, in the range of 1 to 5, in the range of 1 to 4, in the range of 1 to 3, or 2, or 1.

A BTX-binding aptamer structure may comprise full base pairing or partial base pairing. Full base pairing is when there is A-T (adenine-to-thymine) and/or G-C (guanine-to-cytosine) pairing in a stem. Partial base pairing can occur when there are mismatches in a stem structure, such as G-T (guanine-to-thymine) mismatches, along with A-T (adenine-to-thymine) and/or G-C (guanine-to-cytosine) pairing, and thus a BTX-binding aptamer structure may comprise nucleotide mismatching in stem structures. Further, an aptamer may have one or more stems with one or more nucleotide bulges in the stem. A nucleotide bulge may be a single nucleotide bulge or a multiple nucleotide bulge having, for example, 2, 3, 4, or 5 nucleotides.

Additionally, a BTX-binding aptamer may have one or more unpaired nucleotide region in the form of a loop extending from a stem, e.g., 1, 2, 3, 4, or 5 loops. In the aptamer, the one or more loop(s) may have a nucleotide length in the range of 3 to 30 nucleotides, 3 to 25 nucleotides, 3 to 20 nucleotides, 3 to 15 nucleotides, 3 to 10 nucleotides, 3 to 8 nucleotides, or 3 to 6 nucleotides. However, additional or fewer loops and bulges may be including in a BTX-binding aptamer according to some example embodiments.

As mentioned above, a BTX-binding aptamer, according to some example embodiments, may be DNA, RNA, or XNA molecule. A DNA-based BTX-binding aptamer may offer an advantage of being, for example, chemically stable and relatively inexpensive to produce. An RNA-based BTX-binding aptamer may be implemented with a wider variation on three-dimensional structures relative to a DNA aptamer. Alternatively, a BTX-binding aptamer formed from XNA, which may be a synthetic nucleic acid analog with a different sugar backbone than the natural nucleic acids DNA and RNA. Such synthetic nucleic acid analogs may include 1,5-anhydrohexitol nucleic acid (HNA), cyclohexene nucleic acid (CeNA), threose nucleic acid (TNA), glycol nucleic acid (GNA), locked nucleic acid (LNA) peptide nucleic acid (PNA), FANA (fluoro arabino nucleic acid), or the like.

According to some example embodiments, chemically modified, non-natural nucleotides that are resistant to degradation can be used to make the BTX-binding aptamer. Such non-natural nucleotides may include sugar-modified cations of nucleoside triphosphates, which may increase the resistance of the aptamer to nucleases or other enzymes. In this regard, other modifications to provide nuclease resistance to the aptamers may include using locked nucleic acids (LNAs), 2′-O-methylation, 2′-fluorination, 2′-amination, phosphorothiolation, and 3′-capping, which may improve the aptamer's stability.

According to some example embodiments, the BTX-binding aptamer may be component of a BTX-binding molecule. Additionally, the BTX-binding molecule may comprise a detection component. According to some example embodiments, the detection component may be a molecule that is linked to BTX-binding aptamer. The detection component may have a detection property that facilitates detection of the detection component, and thus the aptamer when bound to, for example, a BTX antigen. The detection property may be dynamic such that the detection property is activated in response to the BTX-binding aptamer binding to a BTX antigen. For example, according to some example embodiments, because the BTX-binding aptamer may change structure upon binding to the BTX antigen, the change in structure of the BTX-binding aptamer may cause the detection property to be activated. Alternatively, due to the binding of the BTX-binding aptamer to the BTX antigen, the detection component may be subject to a reaction or change in linking that causes the detection property to be activated. As such, according to some example embodiments, the detection property may be in a first state (e.g., dormant state) when the BTX-binding aptamer of the BTX-binding molecule is not bound to an antigen, and the detection property may transition into a second state (e.g., an active state) in response to the BTX-binding aptamer being bound to an antigen.

According to some example embodiments, a BTX-binding molecule may comprise a BTX-binding aptamer that may be conjugated to a non-nucleotide component, such as a polymeric material (e.g., polyethylene glycol (PEG), polypropylene oxide (PPO), or polyethylene oxide (PEO), or the like). A higher molecular weight compound, like PEG, PPO, and PEO, may increase the stability of the BTX-binding molecule. According to some example embodiments, the non-nucleotide component may be, for example, a nanomaterial or a nanoparticle.

According to some example embodiments, the BTX-binding aptamer may comprise a structure that can transform or switch when the BTX-binding aptamer interacts, for example, with a target BTX antigen. In this regard, prior to interacting with a target BTX antigen, the BTX-binding aptamer may have a first structure (or first conformation), and upon interaction and binding with the target BTX antigen, the structure of the BTX-binding aptamer may change to a second structure (or second confirmation). According to some example embodiments, this change in structure of the BTX-binding aptamer may be leveraged for implementation within a BTX-binding molecule for detection since the change in structure may move components or moieties of the BTX-binding molecule closer or further from each other to effectuate changes in behavior of the BTX-binding molecule.

According to some example embodiments, the BTX-binding molecule may include a linker component that may be a linker molecule. The linker component may be covalently linked to the BTX-binding aptamer or non-covalently linked to the BTX-binding aptamer. Further, for example, linker components, such as non-nucleotide component linker components, for BTX-binding aptamers may be cleavable or non-cleavable, depending on an application for which the linked moiety may be used. In some example embodiments, a linker component may comprise a polymeric material that may employ, for example, polyethylene glycol links to link the BTX-binding aptamer to another molecule, such as a detection component (e.g., a detection reagent). In some embodiments, a bifunctional crosslinker may embody or comprise the linker component to link the BTX-binding aptamer, directly to indirectly, to a detection component. According to some example embodiments, the linker component may use a bifunctional crosslinker that employs an 1-Ethyl-3-[3-dimethylaminopropyl]carbodiimide hydrochloride (EDC)/N-hydroxysulfosuccinimide (sulfo-NHS) coupling chemistry. Other specific types of linker components may be include fatty acids and pH-cleavable linkers such as, for example, an acetal linker (e.g., 3,9-bis(3-aminopropyl)-2,4,8,10-tetraoxaspiro [5.5]undecane (ATU)), a GSH-reducible linker, a photocleavable linker, an acid-labile hydrazone linker, a cathepsin B-labile valine-citrulline dipeptide linker, or a disulfide-based, traceless cleavage linker, such as, 4-nitrophenyl 4-(2-pyridyldithio) benzyl carbonate (NPDBC) or 4-nitrophenyl 2-(2-pyridyldithio) ethyl carbonate (NPDEC). Coupling the BTX-binding aptamer to the desired moiety may also be accomplished by modifying the BTX-binding aptamer with, for example, phosphorothioate (PS) at a desired position on the backbone of the BTX-binding aptamer.

According to some example embodiments, a non-nucleotide moiety such as a detection component in the form of a detection reagent may be chemically linked to strand's 3′- or 5′-terminus of the BTX-binding aptamer. To do so, according to some example embodiments, a terminus of, for example, a DNA strand may be modified with an active thiol or primary amine to provide a conjugate with the desired moiety. Further, the BTX-binding aptamers described herein may also be attached to biotin, desthiobiotin, digoxigenin, or other detection components, for example, at 3′-end or 5′-end of the aptamer for use as detection reagents to label the aptamer.

According to some example embodiments, an aptamer, such as a BTX-binding aptamer, can be formed by complementary nucleic acid base pairing, which can create secondary structures, for example, a short helical arm and a single-stranded (unpaired) loop. A structure (e.g., tertiary structure) of an aptamer may result in a combination of secondary structures, folding in a way that can result in antigen binding for the aptamer. The antigen binding of the aptamer, due to the folding, may be caused by van der Waals forces, hydrogen bonding, electrostatic interaction, or the like. In the structure, according to some example embodiments, generally, most of the BTX-binding aptamer, or all of the BTX-binding aptamer, may fold into a stable complex structure capable of BTX antigen interaction and binding.

According to some example embodiments, a BTX-binding aptamer may be, for example, 15 to 200 nucleotides in length. For example, a BTX-binding aptamer may have a length of at least about 20 nucleotides, at least about 25 nucleotides, at least about 30 nucleotides, at least about 35 nucleotides, at least about 40 nucleotides, at least about 45 nucleotides, at least about 50 nucleotides, at least about 55 nucleotides, or at least about 60 nucleotides. According to some example embodiments, a BTX-binding aptamer may have a length of up to about 200 nucleotides, up to about 150 nucleotides, up to about 100 nucleotides, up to about 90 nucleotides, up to about 85 nucleotides, up to about 80 nucleotides, up to about 75 nucleotides, or up to about 70 nucleotides. According to some example embodiments, a BTX-binding aptamer may have a length within a range of any of the numerical values set forth herein, for example, such as in the range of about 15 to about 200 nucleotides, about 25 to about 150 nucleotides, about 35 to about 100 nucleotides, about 40 to about 90 nucleotides, about 45 to about 80 nucleotides, about 50 to about 75 nucleotides, or about 55 to about 70 nucleotides. However, other lengths may be included as well in other examples.

According to some example embodiments, the BTX-binding aptamer of the BTX-binding molecule or another aptamer of the BTX-binding molecule may target a nucleic acid sequence that is unrelated to the target BTX antigen. In this regard, the nucleic acid sequence for the BTX-binding aptamer may not be responsible for forming a structure (e.g., a tertiary structure) for binding to the target BTX antigen, but the structure of the BTX-binding aptamer may, for example, interact with or bind to another target associated with a brevetoxin. For example, the target for the BTX-binding aptamer may be another oligonucleotide or portion of a nucleic acid that is complementary to the nucleic acid sequence of the BTX-binding aptamer that could nonetheless be used for detection. For example, a target nucleic acid for the BTX-binding aptamer may be coupled to a detectable moiety, such as a fluorophore, that is related to or otherwise associated with a BTX antigen of interest.

According to some example embodiments, the term “detectable moiety” (also known as a “label”) refers to a moiety capable of being detected by an analytical technique. Exemplary labels include radioisotopes, mass tags, fluorescent labels/fluorophores, luminescent groups, phosphorescent groups, or the like. Such labels may operate as signal-generating reporter groups that can be detected without further modifications.

In this regard, for example, radioisotopes may include tritium, 32P, 33P, 35S, 14C, or the like. Fluorescent labels/fluorophores (“fluorescent dyes”) may include molecules that absorb light energy at a defined excitation wavelength and emit light energy at a different wavelength. Examples include, but are not limited to: Alexa Fluor dyes (Alexa Fluor 350, Alexa Fluor 488, Alexa Fluor 532, Alexa Fluor 546, Alexa Fluor 568, Alexa Fluor 594, Alexa Fluor 633, Alexa Fluor 660 and Alexa Fluor 680), AMCA, AMCA-S, BODIPY dyes (BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665), Carboxy-rhodamine 6G, carboxy-X-rhodamine (ROX), Cascade Blue, Cascade Yellow, Coumarin 343, Cyanine dyes (Cy3, Cy5, Cy3.5, Cy5.5), Dansyl, Dapoxyl, Dialkylaminocoumarin, 4′,5′-Dichloro-2′,7′-dimethoxy-fluorescein, DM-NERF, Eosin, Erythrosin, Fluorescein, FAM, Hydroxy-40 coumarin, IRDyes (IRD40, IRD 700, IRD 800), JOE, Lissamine rhodamine B, Marina Blue, Methoxycoumarin, Naphthofluorescein, Oregon Green 488, Oregon Green 500, Oregon Green 514, Pacific Blue, PyMPO, Pyrene, Rhodamine B, Rhodamine 6G, Rhodamine Green, Rhodamine Red, Rhodol Green, 2′,4′,5′,7′-Tetra-bromosulfone-fluorescein, Tetramethyl-rhodamine (TMR), Carboxytetramethyl-rhodamine (TAMRA), Texas Red, and Texas Red-X.

A “mass tag” may refer to any moiety that can be uniquely detected by the moiety's mass using mass spectrometry (MS) detection techniques. Examples of mass tags include electrophore release tags. See, for example, U.S. Pat. Nos. 4,650,750 and 5,650,270, which are incorporated herein at least for their disclosure of electrophore release tags.

Example embodiments of a BTX-binding molecule as provide herein, may also include a “secondary label”. Such second label may be associated with a second moiety, such as a biotin or various protein antigens that may require the presence of a second intermediate to produce a detectable signal. For example, for biotin, the secondary intermediate may include streptavidin-enzyme conjugates. For example, for antigen labels, the secondary intermediate may include antibody-enzyme conjugate.

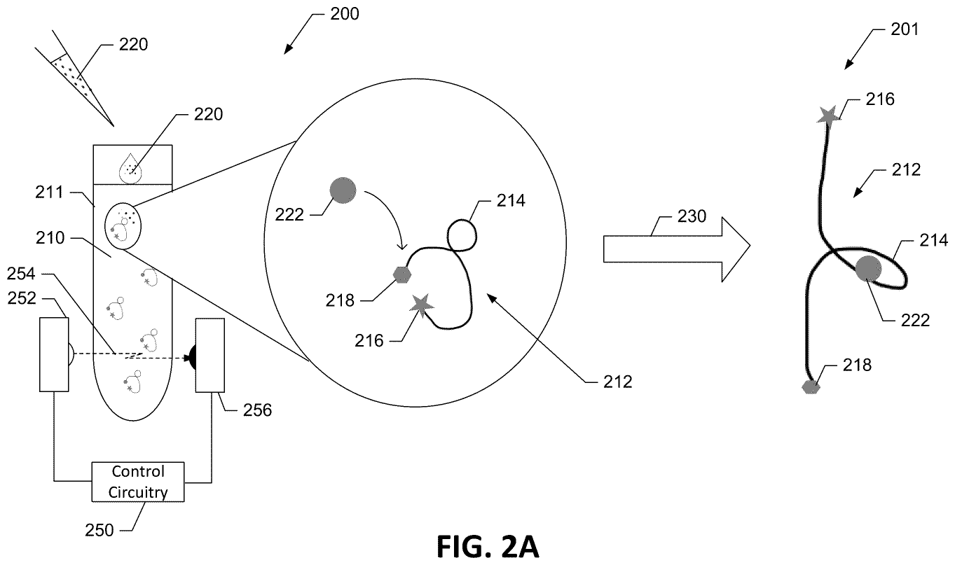

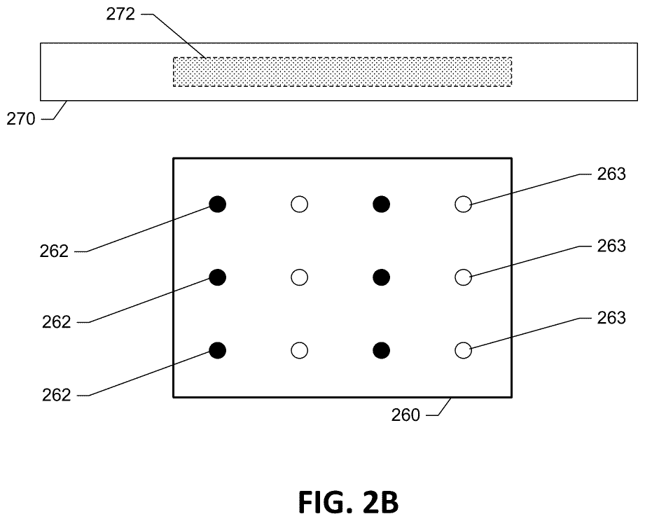

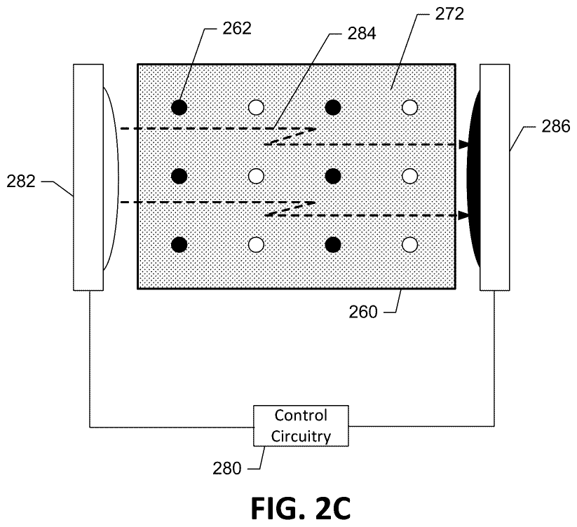

Accordingly, a BTX-binding molecule may be implemented as a component of a detection device or method as described herein. According to some example embodiments, a BTX-binding molecule comprising a BTX-binding aptamer may be employed in devices, systems, and methods for BTX detection. For example, as shown in FIG. 2, a BTX detection may be performed via implementation of a fluorescent aptamer assay (FAA) using a detector solution 210 that comprises a BTX-binding molecule 212. Detection of A- and B-type BTX via the FAA may be performed based on the signal transduction principle of fluorescence resonance energy transfer (FRET).

The BTX detection of FIG. 2A involves a sample 220 being applied to a detector solution 210 for the FAA. The sample 220 may comprise BTX 222 from, for example, shellfish, water samples, other aquatic samples, or the like. The detector solution 210 may comprise a plurality of BTX-binding molecules 212 within, for example, a solvent, and may be disposed within holder 211 (e.g., a test tube, slide, Petri dish, absorbent media, substrate, or the like). A BTX-binding molecule 212, as shown in the zoom circle, may comprise a BTX-binding aptamer 214 operably coupled to a fluorophore 216 and a fluorescent quencher 218 (e.g., an oligonucleotide-labeled quencher). Collectively, the fluorophore 216 and the fluorescent quencher 218 may embody a detector component of the BTX-binding molecule 212, and the operation of the fluorophore 216 and fluorescent quencher 218 may embody a detection property of the detector component. According to some example embodiments, the BTX-binding aptamer 214 may comprise, for example, a first stem that links the BTX-binding aptamer 214 to the fluorophore 216 and a second stem that links the BTX-binding aptamer 214 to the fluorescent quencher 218. The BTX-binding aptamer 214, prior to being bound to a target BTX antigen, may have a first structure where the stems fold in a manner that places the fluorophore 216 in close proximity to the fluorescent quencher 218. As such, the fluorescent quencher 218 may be close enough to the fluorophore 216, when the BTX-binding aptamer 214 is in an unbound, first structural state, to absorb energy from the fluorophore 210 and render the detector solution 210 non-fluorescent, as seen at 200. However, when the BTX-binding aptamer 214 interacts with and binds to a BTX antigen of the BTX 222, the binding causes the structure of the BTX-binding aptamer 214 to change which displaces the fluorophore 216 away from the fluorescent quencher 218 as shown in 201 thereby activating the detection property of the fluorophore 216 and fluorescent quencher 218. Due to this displacement, the fluorescent quencher 218 may no longer absorb the energy from the fluorophore 216, and therefore a florescent signal may be generated that is related to, or a function of, the binding of the BTX-binding aptamer 214 to the BTX 22.

From a procedural perspective, at 200, the sample 220 may be applied to the detector solution 210. Prior to binding, as described above, the structure of the BTX-binding aptamer 214 may cause the fluorescent quencher 218 to prevent the detector solution 210 from fluorescing due to the presence of the fluorophore 216. However, upon interaction and binding of the BTX-binding aptamer 214 with the BTX 222, as indicated by arrow 230, the BTX-binding aptamer 214 is reconfigured by the binding to the BTX 222 into a different structure where the florescence generated by the fluorophore 216 is no longer quenched by the fluorescent quencher 218, due the fluorophore 216 being moved a sufficient distance away from the fluorescent quencher 218 or being freed from the BTX-binding aptamer 214. As such, a detectable fluorescent signal may be generated by the detector solution 210 that can be indicative of the presence of BTX 222 in the sample 220 and the concentration of BTX molecules within the sample 220.

To detect the fluorescent signal, an illumination device 252 (e.g., black light) may be controlled, for example, by control circuitry 250, to output light illumination 254 at a desire wavelength (e.g., ultraviolet wavelengths) and intensity. The illumination 254 may cause the fluorescent signal generated by the detector solution 210 to be detectable (e.g., activated to be, for example, visible) to a light detector 256. According to some example embodiments, the illumination 254 may pass through the detector solution 210, now mixed with the sample 220, to a light detector 256. One of skill in the art would appreciate that the configuration of the illumination device 252 and the light detector 256 is but one simplified configuration and that other configurations involving, for examples, lenses and mirrors may be used. The light detector 256 may be configured to detect, for example, the presence of a fluorescent signal, a wavelength of the fluorescent signal, or an intensity of the fluorescent signal. The control circuitry 250 may be configured to receive a detection signal from the light detector 256. The control circuitry 250 may also be configured to evaluate, for example, the presence, wavelength, or intensity information in the detection signal, based on the input wavelength and intensity from the illuminator device 252, and determine whether BTX 222 is present in the sample 220. Additionally, according to some example embodiments, the control circuitry 250 may be configured to evaluate, for example, the presence, wavelength, or intensity information in the detection signal, based on the input wavelength and intensity from the illuminator device 252, and determine a concentration of molecules of BTX 222 in the sample 220.

FIGS. 2B and 2C illustrate another example embodiment of a detection device and process according to some example embodiments. In this regard, a plate 260 having immobilized test samples 262 and immobilized control samples 263 are utilized. A detector solution 272 may be applied to the plate 260 and, due to the type of detector solution as further described below, the test samples 262 and the control samples 263 may “compete” for aptamer binding for the purpose of BTX detection. According to some example embodiments, the detector solution 272 may be applied to the plate 262 in any number of ways such as, for example, a bath, a spray applicator, or the like. In the example embodiment shown in FIG. 2B, a reservoir of detector solution 272 is disposed within an applicator device 270 that applies (e.g., via spraying) onto the plate 260.

Now referring to FIG. 2C, the detector solution 272 has been applied and the BTX-binding molecules within the detector solution 272 may have bound to test samples 262 or the control samples 263, thereby allowing for detection of BTXs within the test samples 262. For example, the detector solution 272 may cause a change in light response at the test samples 262 and the control samples 263. Therefore, similar to the approach described above, the plate 260 may be illuminated and a detector may, for example, detect a light response from the plate 260 that is indicative of the presence of a BTX, and possibly a type of BTX, disposed on the plate 260.

In this regard, to detect a light response signal off of the plate 260, an illumination device 282 may be controlled, for example, by control circuitry 280, to output light illumination 284 at a desire wavelength and intensity. The illumination 284 may cause the light response signal generated by the detector solution 272 to be detectable (e.g., activated to be, for example, visible) to a light detector 286. According to some example embodiments, the illumination 284 may reflect off of the plate 260 with the detector solution 272 having been applied, to be received at the light detector 286. The light detector 286 may be configured to detect, for example, the presence of a light response signal, a wavelength of the light response signal, or an intensity of the light response signal. The control circuitry 280 may be configured to receive a detection signal from the light detector 286. The control circuitry 280 may also be configured to evaluate, for example, the presence, wavelength, or intensity information in the detection signal, based on the input wavelength and intensity from the illuminator device 282, and determine whether test samples 262 comprise BTX. Additionally, according to some example embodiments, the control circuitry 280 may be configured to evaluate, for example, the presence, wavelength, or intensity information in the detection signal, based on the input wavelength and intensity from the illuminator device 282, and determine a concentration of molecules of BTX on the plate 260.

According to some example embodiments, implementation of the process performed in FIGS. 2B and 2C may be an enzyme-linked aptasorbent assay (ELASA). ELASA may involve implementation of an indirect competitive assay where plate-immobilized test samples 262 compete against control samples 263, which may have a known BTX, for binding to a biotin-labeled BTX-binding aptamer in the BTX-binding molecule of the detector solution 272. In this example method, higher concentrations of BTXs in the control samples 263 may result in fewer free BTX-binding aptamers in the detector solution 272 binding to the plate 260. BTX-binding aptamers that do bind to the plate 260 may be quantified using, for example, a streptavidin-conjugated reporter enzyme that may be included in the BTX-binding molecule. The reporter enzyme may change the light signal response due the binding of the BTX-binding aptamers in the detector solution 272. Further, according to some example embodiments, a substrate hydrolysis operation may be performed on the plate 260, involving illumination and detection, which may yield a signal having a known characteristic, such as, for example, being inversely proportional to a BTX concentration, in a sample used for the test samples 262. In this regard, according to some example embodiments, once a microwell is coated with conjugated BTX, the assay may be completed in, for example, less than 2 hours.

Using high affinity sequences in BTX-binding aptamers for use in BTX detection, methods, devices, and systems can be developed based on the teachings provided herein. While an FAA and an ELASA process and device have been discussed, it is understood that additional or different assays and tests may be implemented in a similar manner. Such additional assays and tests may include, for example, Aptamer-Based Lateral Flow Assays.

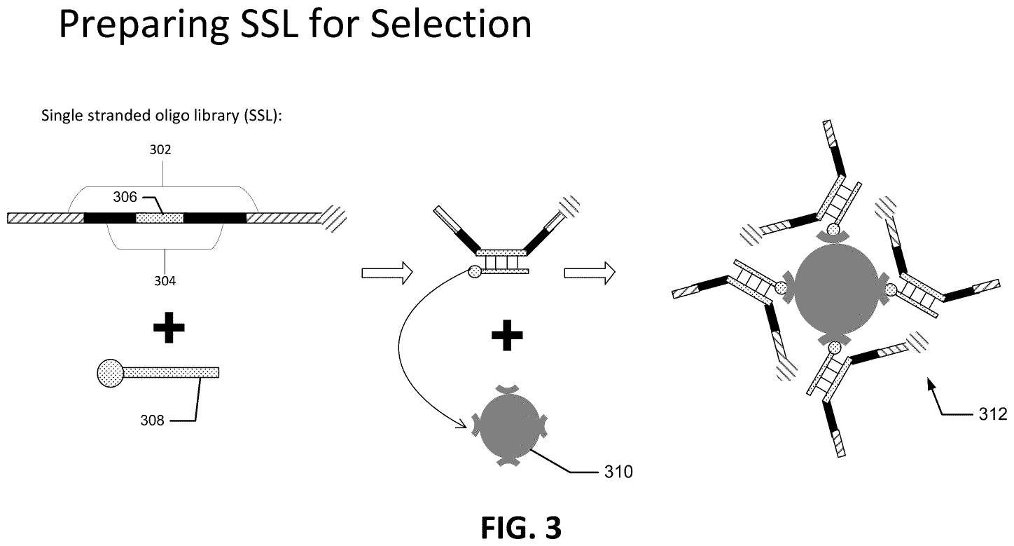

Referring back to the determination and development of a BTX-binding aptamer, another example SELEX procedure may be performed. In this regard, the following procedure was implemented and is described here to provide further support and teachings of the BTX-binding aptamers. In this regard, the example SELEX procedure was carried out to identify and create BTX-binding aptamers. A structure-switch SELEX method was carried out to select for aptamer sequences which changed conformation and were displaced from a bead-adhered complementary nucleotide strand in the presence of BTX targets. FIG. 3 shows a starting single stranded library (SSL) consisting of constant primer regions 302 at either end, followed by random nucleotide stretches 304 and a central constant region 306 for a binding complementary sequence 308 (CSSL). A FAM labelled forward primer was used in PCR to amplify and regenerate a fluorescent SSL following a selection cycle. A poly A tagged reverse primer was also used to yield an anti-sense strand which could separate by size from the SSL following PCR amplification.

More generally, initial SSL of sequences, from which BTX sequences could be derived, included constant regions for PCR amplification, random regions of sequences for diversity purposes, and a central constant region for adhesion to a complementary strand on a bead. Displacement may be sensitively detected through a fluorescent FAM label on the sequences.

As illustrated in FIG. 3, for each selection cycle, an SSL and CSSL were annealed together and subsequently adhered to an agarose bead 310 through a strong biotin-streptavidin interaction to assemble a complex 312. The beads could then be spun down into a pellet to allow for easy physical separation of displaced sequences from those remaining sequences.

In a random sequence library, sequences exist which naturally displace from a complementary strand more easily as well as sequences that are not desirable for a final aptamer to interact with. As a result, negative selection was first performed in each selection cycle to wash away and eliminate sequences which naturally displace from the beads over a typical time period of the example SELEX process. For example, in a BTX selection design, negative selection was performed by incubating 400 μL of selection buffer with 100 μL of SSL-coated beads (1 nmol of SSL can be used to coat the beads in the initial cycle, followed by >0.1 nmol in subsequent cycles). The beads could then be spun down and fluorescence of negatively selected sequences in a supernatant measured.

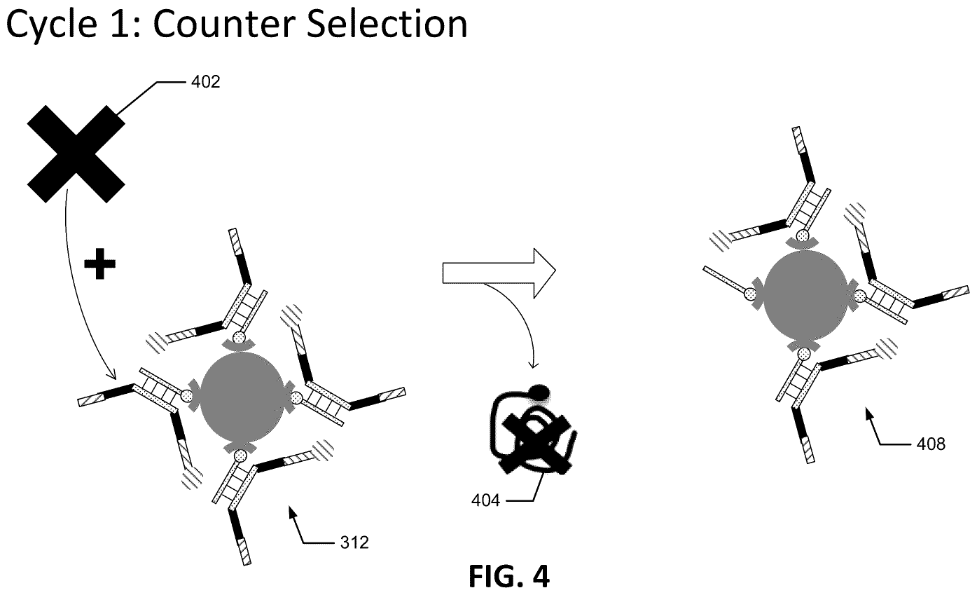

As illustrated in FIG. 4, a counter selection was also performed in most cycles to remove or eliminate sequences displaced by undesirable targets, e.g., saxitoxin dihydrochloride, okadaic acid, and domoic acid, which might interfere with BTX detection. Supernatant fluorescence from a given cycle may indicate an extent of sequence displacement by factors other than BTXs. Counter selections may generally be performed by mixing 100 μL of washed negatively selected beads with 400 μL of selection buffer and 20 μL total counter target volume. After which, the beads were spun down and fluorescence of counter selected sequences in supernatant measured. In FIG. 4, counter target 402 is shown to be added to the complex 312 and oligonucleotides 404 that were displaced by counter target 402 are removed in supernatant. Complex 408 remains, as the beads with oligonucleotides that did not bind to counter target 402 are collected.

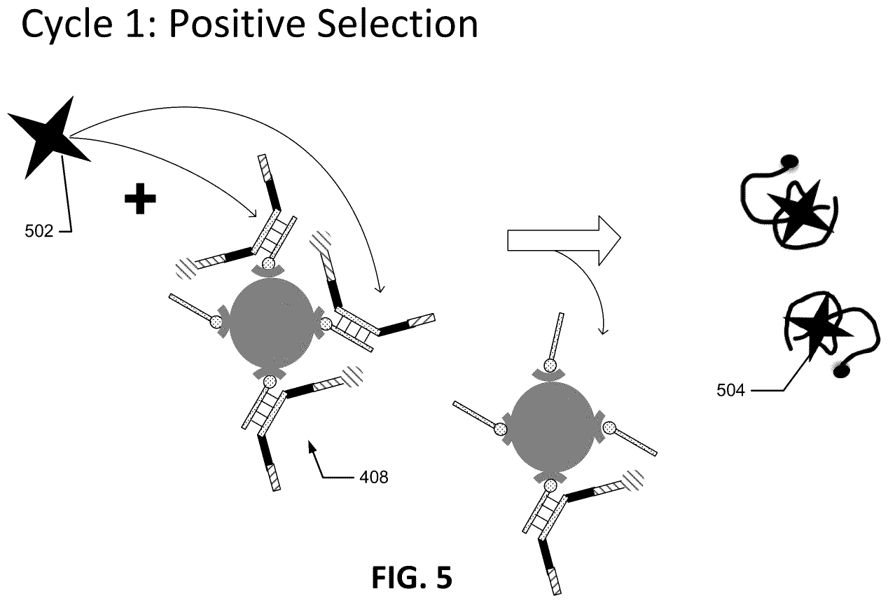

Finally, positive selection was carried out to select for BTX-interacting potential aptamer sequences as shown in FIG. 5. In one example, BTX 1, BTX 2 (open A ring), and BTX 3 were selected to represent the main molecular backbones or positive target panel. However, other BTX molecules could be used as well. In one example, all these backbones should interact with a final aptamer. Positive selection was typically be carried out by mixing 100 μL of washed counter selected beads with 400 μL of selection buffer and 10 μL total positive target volume. Next, the beads were spun down and fluorescence of the positively selected sequences in supernatant measured to indicate an extent of sequence displacement by target BTXs. In FIG. 5, brevetoxin 502 is shown to be added to the complex 408 and beads with oligonucleotides that did not bind to brevetoxin 502 are discarded. Oligonucleotides 504 are therefore enriched for brevetoxin binding sequences.

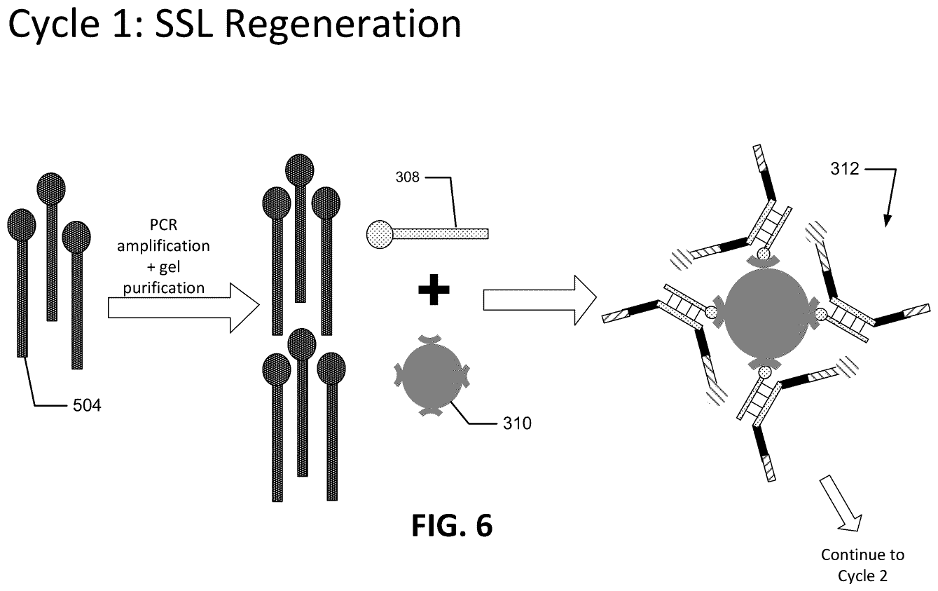

As shown in FIG. 6, relative amounts of SSL released from each kind of selection were determined through fluorescence, and the BTX-interacting enriched SSL pool amplified through PCR and purified through poly acrylamide gel electrophoresis (PAGE) and ethanol precipitation to then undergo further selection cycles. Over rounds of selection, a positive fluorescent signal increased relative to negative and counter fluorescence, suggesting that more of the pool was interacting with the intended target. More generally, FIG. 6 depicts single-stranded library (SSL) regeneration. Following a selection cycle, surviving BTX-interacting sequences were amplified, purified, and coated to beads for further evolution cycles.

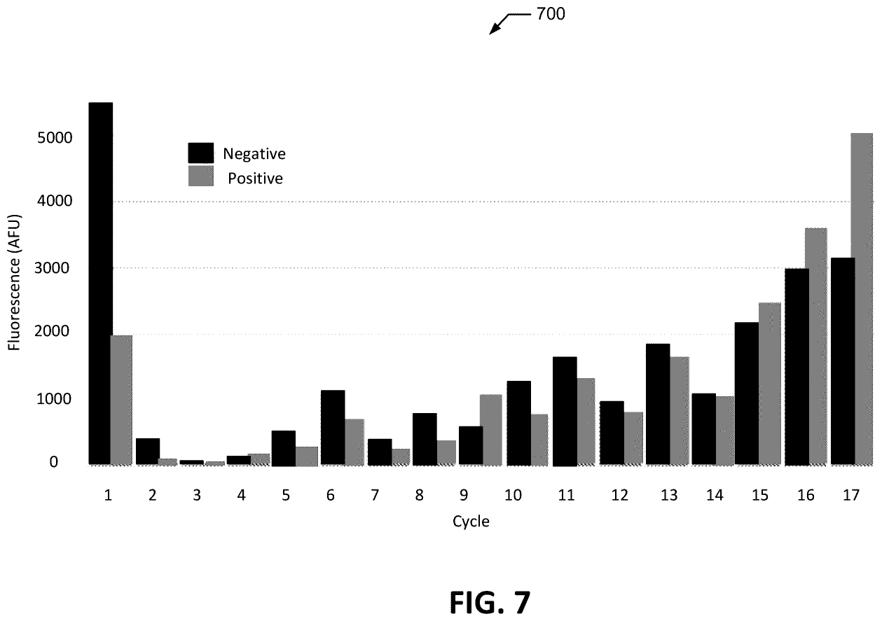

In turn, fluorescent results over successive BTX selection cycles were obtained as shown in the graph 700 of FIG. 7. As illustrated, cycle 1 fluorescent results indicated that there were many more sequences not-specifically released from the agarose beads than were released by BTX targets. The signal for the following initial cycles was very low, indicating a release of few sequences. Over successive cycles, which included alterations to selection parameters such as washing steps, incubation time, and inclusion or exclusion of the counter selection, the signal gradually increased overall.

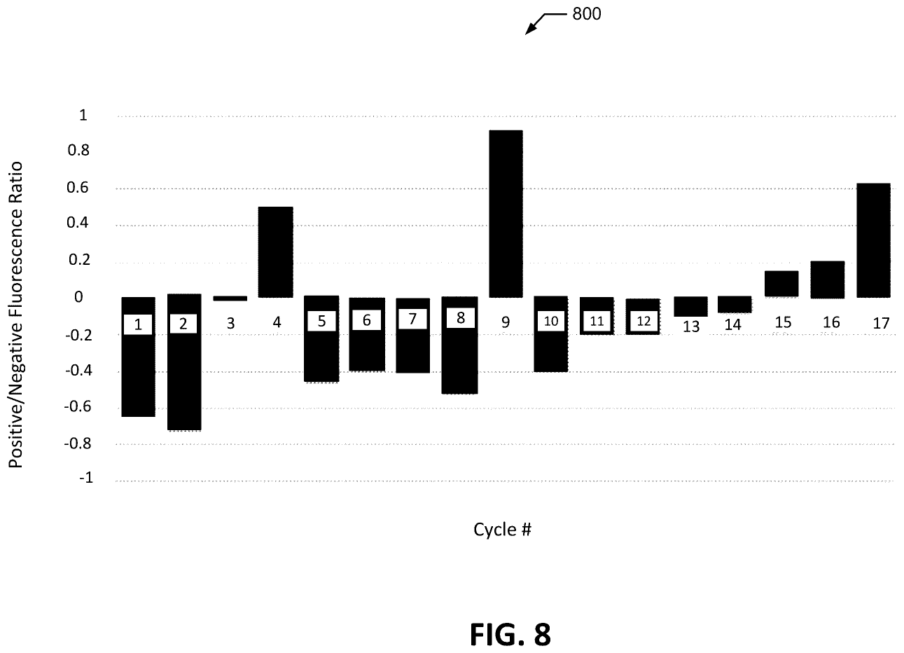

FIG. 8 illustratively shows a graph 800 of a ratio of positive to negative fluorescence at each BTX selection cycle. As shown, a ratio of positive to negative fluorescence increased overall as more of the sequence population demonstrated BTX interaction, with a final positive signal 1.6× the negative.

After a fair positive to negative signal was generated, the collected positively selected material was sequenced. The top 50 most common sequences in the library found post BTX SELEX are provided in the following Table 2. The example constant starting primer (ATCGCACTGACAGCT) and the example ending primer (TCATCGCATCGCATCA) parts of the sequences are not shown for simplicity. BTXAP01, BTXAP02, BTXAP03, etc. correspond to SEQ ID NO:1, SEQ ID NO:2, SEQ ID NO:3, etc.

TABLE 2

| NUMBER | SEQUENCE |
|---|---|
| BTXAP01 | CACCAACGGTACTAAATGCATGTACGATAATGTCTCT |
| | CCCGCTCTGTA |
| BTXAP02 | CACCACAGACCAAAAATGCATGTTACGATAGCTTAAC |
| | ACCTTGCCCTGG |
| BTXAP03 | CACGAAGGGCGCGTAATGCATGTTACGATTATTATCC |
| | CCCCTTCTGTGG |
| BTXAP04 | CGGGAAGGGCGGTAAATGCATGCACGATTTGTATCTA |
| | ACCTCCTCATGG |
| BTXAP05 | CACCAAACGAAAAGAATGCATGTTACGATTATTCGCA |
| | CTCGCCTCTGGG |
| BTXAP06 | CACCGAACGGAACACATGCATGTTACGATTAACTCCA |
| | CCCAACTCGTGG |
| BTXAP07 | CAAGGGGGGGTGGAAATGCATCTACGATAATCATCTC |
| | CGCTTCCCGTG |
| BTXAP08 | CACCGAAGAGGAGATATGCATGTTACGATCCCTTAGT |
| | ATCTATTGCTTG |
| BTXAP09 | CACAACCGGGCGTAAATGCATGCTAGATATGTTCTTG |
| | TCTACTCTTGG |
| BTXAP10 | CACGACCAGCCCCAAATGCATGTTACGATTTGTACAC |
| | TCACGCACTTTG |
| BTXAP11 | ACACAACGGGTTTAAATGCATGTTACGATACTTCAGG |
| | CTATTCCCTTGG |
| BTXAP12 | CACGAAGGGCAAATTATGCATGCACGATCTTACCGTT |
| | CTCACCTTCCG |
| BTXAP13 | ACCACGGCAGAGGTAATGCATGTTACGATTCAAATCA |
| | TCCCCTTTGGTG |
| BTXAP14 | CGCAAAGGGGGACATATGCATGCTACGATAAGTAGCA |
| | TCCCCTCTCTGG |
| BTXAP15 | CACCCCGCAGACGAAATGCATGTTACGATTTCTCCAA |
| | CTGCACTCTTGG |
| BTXAP16 | CACGGGAAGAACACAATGCATGCACGATATGCAGCCT |
| | TTACACCTTCG |
| BTXAP17 | CACAACGGGCGACAAATGCATGTTACGATTGTATTGA |
| | ACTCCTGACACA |
| BTXAP18 | CACAGCGTAGGACCTATGCATCTACGATAATATCCTA |
| | TACTGCTCTTG |
| BTXAP19 | CACAGGGGCGCATCTATGCATCTTACGATTAAGTCCA |
| | CTTCACCCTTCG |
| BTXAP20 | ACCCGGATCGCCAAAATGCATGTTACGATAGTCAATC |
| | ATTCCGCTCTTG |
| BTXAP21 | ACCAAGGGAAGGATCATGTATGCTACGATACTCTCTC |
| | GCTACCTTCTCG |
| BTXAP22 | CACCGGCAGGTCATCATGTATCCTACGATTAGCTATA |
| | CTACACGCCTTG |
| BTXAP23 | CGAGAAGGAAGGGAAATGCATGTACGATAATCACGCA |
| | CACCCCTCTTG |
| BTXAP24 | CAACCGGAGGATAACATCCATGTTACGATACCCCAAT |
| | GCTCTCCTTGTG |
| BTXAP25 | CGGGAAGGGCTACCGATGTATGCTACGATTCATCACT |
| | CACTCGTCCCCA |
| BTXAP26 | CCACGAAGCACAAACATCCATGTTACGATATGTCAAC |
| | ACGCCTTTCTCG |
| BTXAP27 | CGACAAGAGAAGAGAATGCATGTTACGATCCCTACTT |
| | TTCTAACCCTGG |
| BTXAP28 | CACCGAAGGGCCAGAATGCATGTTACGATAGGACTTT |
| | TATTTCCCTTCG |
| BTXAP29 | CACGACAGGCCTAAATGCATGGCTACGATAAGTGGCT |
| | TTATACCCCATG |
| BTXAP30 | CGCAACCAAGAGACAATGCATGTTACGATACACGTTC |
| | ATCCTCCACTTG |
| BTXAP31 | CACCCGAGGGAAGCAATACATGCTACGATAGAAAGTG |
| | TGTCCTTCCTGG |
| BTXAP32 | AACGGCAGACACGCAATGCATGTTACGATAAACCGTA |
| | CATTCTGCCTTG |
| BTXAP33 | ACCCCAACAAGAGAGATGCATGTTACGATAATCAGCC |
| | TATCATACGCCA |
| BTXAP34 | CACCCCAAGTACCATATGCATGCTAGATAAATAGGTT |
| | TCCTTCTTGGG |
| BTXAP35 | CACAGCGGCGTACCCATGCATGTTACGATATCAAGTG |
| | ACATCCTCCGCA |
| BTXAP36 | CACGACATGGACATTATGCATGCTAGATATACTTTCA |
| | GACCTCTCTTG |
| BTXAP37 | CACAACAGGCAGAACATGCATGCTAGATAATCGTTGT |
| | TCACCTCTTGG |
| BTXAP38 | CACCAACGGGTCCAAATGCATGCTAGATACTCTCACA |
| | CTTCACTCGCA |
| BTXAP39 | ACCCGGCAATGGGAAATGCATGTTACGATTATTACTA |
| | CCTTGCTCCGTA |
| BTXAP40 | CACCACGCAAGGGGTATGCATGTTACGATATACTCAC |
| | TAGGCCATCTGG |
| BTXAP41 | ACCCACCAGGAGAGCATGCATGTTACGATATTCTTCG |
| | CAACCTTACGGG |
| BTXAP42 | CGGGAGGAAGACCATATGCATGCTAGATATAATCTGA |
| | CCCTCCACGTG |
| BTXAP43 | CACACCAGGGAACTGATGTATGCTACGATACTTCATC |
| | CTTCCTCTCTGG |
| BTXAP44 | ACCGAAGAGTCAAATATGCATGTTACGATACATTGCT |
| | AACCCCTCTTTG |
| BTXAP45 | CACAACAGGCGTAGTATGCATGTTACGATTCATCAAC |
| | TGCTATTCCTGG |
| BTXAP46 | ACCAGAAGGGCAACAATGAATGCTACGATTAGAATTC |
| | GCCTTTGCTTGG |
| BTXAP47 | CACAAGCAAGCCTAAATGCATGCTAGATAATGAGTAT |
| | CCTCCTTGTGG |
| BTXAP48 | CACAGAAGGGCAGATATGCATGCTAGATCATAGTCAC |
| | TTCCTATCCCG |
| BTXAP49 | CGGGAAGGTGAGGTAATGTATGCTACGATACCCCCAC |
| | GCGCTTCCATCG |
| BTXAP50 | CAACGGAAGACGACAATGCATGCTAGATATCATTTTA |
| | CGCTACCTTCG |

From the top 50 sequences, the top 20 sequences as provided below in Table 3, including constant primer regions shown as underlined, are as follows. Most sequences showed a mutation in the central constant region used to anneal the sequences to the beads. The slight mismatch might have been selected to allow for easier BTX-mediated release of the sequences.

TABLE 3

| BTXAP | ATCGCACTGACAGCT |
|---|---|
| 01 | CACCAACGGTACTAAATGCATGTAC |
| | GATAATGTCTCTCCCGCTCTGTA |
| | TCATCGCATCGCATCA |
| BTXAP | ATCGCACTGACAGCTCACCACAGACCAAAAATGCATGTTA |
| 02 | CGATAGCTTAACACCTTGCCCTGGTCATCGCATCGCATCA |
| BTXAP | ATCGCACTGACAGCTCACGAAGGGCGCGTAATGCATGTTA |
| 03 | CGATTATTATCCCCCCTTCTGTGGTCATCGCATCGCATCA |
| BTXAP | ATCGCACTGACAGCTCGGGAAGGGCGGTAAATGCATGCAC |
| 04 | GATTTGTATCTAACCTCCTCATGGTCATCGCATCGCATCA |
| BTXAP | ATCGCACTGACAGCTCACCAAACGAAAAGAATGCATGTTA |
| 05 | CGATTATTCGCACTCGCCTCTGGGTCATCGCATCGCATCA |
| BTXAP | ATCGCACTGACAGCTCACCGAACGGAACACATGCATGTTA |
| 06 | CGATTAACTCCACCCAACTCGTGGTCATCGCATCGCATCA |
| BTXAP | ATCGCACTGACAGCTCAAGGGCGGGTGGAAATGCATCTAC |
| 07 | GATAATCATCTCCGCTTCCCGTGTCATCGCATCGCATCA |
| BTXAP | ATCGCACTGACAGCTCACCGAAGAGGAGATATGCATGTTA |
| 08 | CGATCCCTTAGTATCTATTGCTTGTCATCSCATCGCATCA |
| BTXAP | ATCGCACTGACAGCTCACAACCGGGCGTAAATGCATGCTA |
| 09 | GATATGTTCTTGTCTACTCTTGGTCATCGCATCGCATCA |
| BTXAP | ATCGCACTGACAGCTCACGACCAGCCCCAAATGCATGTTA |
| 10 | CGATTTGTACACTCACGCACTTTGTCATCGCATCGCATCA |
| BTXAP | ATCGCACTGACAGCTACACAACGGGTTTAAATGCATGTTA |
| 11 | CGATACTTCAGGCTATTCCCTTGGTCATCGCATCGCATCA |
| BTXAP | ATCGCACTGACAGCTCACGAAGGGCAAATTATGCATGCAC |
| 12 | GATCTTACCGTTCTCACCTTCCGTCATCGCATCGCATCA |
| BTXAP | ATCGCACTGACAGCTACCACGGCAGAGGTAATGCATGTTA |
| 13 | CGATTCAAATCATCCCCTTTGGTGTCATCGCATCGCATCA |
| BTXAP | ATCGCACTGACAGCTCGCAAAGGGGGACATATGCATGCTA |
| 14 | CGATAAGTAGCATCCCCTCTCTGGTCATCGCATCGCATCA |
| BTXAP | ATCGCACTGACAGCTCACCCCGCAGACGAAATGCATGTTA |
| 15 | CGATTTCTCCAACTGCACTCTTGGTCATCGCATCGCATCA |
| BTXAP | ATCGCACTGACAGCTCACGGGAAGAACACAATGCATGCAC |
| 16 | GATATGCAGCCTTTACACCTTCGTCATCGCATCGCATCA |
| BTXAP | ATCGCACTGACAGCTCACAACGGGCGACAAATGCATGTTA |
| 17 | CGATTGTATTGAACTCCTGACACATCATCGCATCGCATCA |
| BTXAP | ATCGCACTGACAGCTCACAGCGTAGGACCTATGCATCTAC |
| 18 | GATAATATCCTATACTGCTCTTGTCATCGCATCGCATCA |
| BTXAP | ATCGCACTGACAGCTCACAGGGGGGCATCTATGCATGTTA |
| 19 | CGATTAAGTCCACTTCACCCTTGGTCATCGCATCGCATCA |
| BTXAP | ATCGCACTGACAGCTACCCGGATGGCCAAAATGCATGTTA |
| 20 | CGATAGTCAATCATTCCGCTCTTGTCATCGCATCGCATCA |

Each sequence may be further assessed and analyzed for interactions with different members of the BTX family and placed into suitable detection applications, as discussed below. Additionally, while specific concentrations and solutions have been discussed as part of a SELEX process to identify BTX aptamers, it is expressly contemplated that other concentrations and steps may be implemented as well.

Nucleic acid sequences for exemplary BTX-binding aptamers are shown above. BTX-binding aptamers may have a nucleic acid sequence that is the same (e.g., 100% identity) as the specific aptamer sequences provided herein, e.g. BTX Aptamer Sequences 1-50 (SEQ ID NOs: 1-50) or 1-20 (SEQ ID NOs: 1-20) (collectively, “BTX sequences”), above, or can have a nucleic acid sequence that is not 100% identical (e.g., <100%) to these specifically provided sequences. The identity can be calculated over the entire length of the aptamer sequence, referred to herein as “a global identity.” Sequences with lower identity to the specific aptamer sequences provided herein can be due to one or more nucleotide changes from the specific aptamer sequences and can be referred to as “variant” or “mutant” aptamer sequences. In exemplary embodiments, a variant aptamer sequence may have a number of nucleotide variation(s) in the range of 1 to 24, in the range of 1 to 20, in the range of 1 to 16, in the range of 1 to 12, in the range of 1 to 10, in the range of 1 to 8, in the range of 1 to 6, such as 1, 2, 3, 4, 5, 6, 7, 8, 9, 10, 11, 12, 13, 14, 15, 16, 17, 18, 19, 20, 21, 22, 23, or 24 nucleotide changes (nucleotide variations or mutations) as compared to the full-length specific template sequences of BTX sequences. The percent identity of a variant sequence is determined by the number of nucleotide changes compared to the original (template) aptamer sequence. For example, a variant aptamer sequence having five nucleotide changes as compared to an original (template) aptamer sequence having a length of 62 nucleotides provides a variant with about 92% identity to the original (template) aptamer sequence (57/62).

Percent (%) identity of a nucleotide sequence is the percentage of nucleotide residues that are identical between a full-length nucleotide candidate (e.g., variant) sequence and full-length template (e.g., any of BTX sequences; SEQ ID NOs: 1 to 50) sequence or a selected portion of the candidate and template sequences when the two sequences are aligned. Percent identity can be determined by aligning sequences and, if necessary, introducing gaps for best alignment to achieve the maximum percent sequence identity. Bioinformatic computer programs such as BLAST, BLAST2, ALIGN2, or Megalign (DNASTAR) can align sequences. Parameters may be provided for measuring alignment, including any algorithms needed to achieve maximal alignment over the length of the sequences being compared.

According to some example embodiments, BTX-binding aptamer variants may have nucleotide sequences that are at least 60% or greater, 65% or greater, 70% or greater, 75% identical, at least 76% identical, at least 77% identical, at least 78% identical, at least 79% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, or at least 99% identical to any one of BTX sequences (SEQ ID NOs: 1 to 50).

Based on the current disclosure and the knowledge in the art regarding aptamers, it will be appreciated that certain regions of the aptamer may be more robust with respect to nucleic acid substitution. For example, variations in stem regions of a secondary or tertiary structure of an aptamer may display less impact on target molecule binding relative to unpaired regions that form an antigen biding pocket, and accordingly a variant BTX-binding aptamer may have substitutions that provide a low degree of identity to an original aptamer sequence, or even no identity in these regions. However, regions of an aptamer that have a secondary or tertiary structure that provides binding to a target antigen may be more sensitive to variation. As such, these regions (e.g., unpaired regions) may have fewer substitutions, additions, or deletions as compared to stem regions. These binding regions can have unpaired nucleic acid bases that form a binding pocket for binding of the target antigen. An unbound nucleic acid pocket can appear in a folding program, or can be recognized by its sequences without using a folding program.

In view of this, variant BTX-binding aptamer sequences may be described in terms of “local” identity to portions of the original aptamer sequence (any one of BTX sequences). For example, there can be one or more nucleotide variations in one or more “regions” of the aptamer, with such regions corresponding to specific 2D structures of the sequence, such as regions of base-pairing (stems), bulges in the stem regions (unpaired nucleotides), and regions of unpaired nucleotide stretches, such as loops extending from a stem region, unpaired nucleotide stretches between stems, and unpaired regions at 5′ and 3′ ends of the aptamer sequence. These regions are identified as sub-sequences of the full-length aptamer sequence and can be described in terms of nucleotide positions in the aptamer, in a 5′ to 3′ direction.

Computational methods for predicting nucleic acid secondary structure may also be used, according to some example embodiments. Tools to determine the secondary structure of DNA such as Mfold, RNAfold, and CentroidFold, among others, can be used to predict the two-dimensional structures of BTX-binding aptamer sequences (SEQ ID NOs: 1 to 50) provided herein. Using a suitable computational method, the secondary structures of the aptamers can be understood, and regions of the aptamers such as base-paired stems, loops, unpaired non-loop regions, and bulges, can be identified for any aptamer species.

For example, a variant aptamer can be referred to with reference to specific nucleotide stretches of BTX sequences as described herein, with those specific stretches corresponding to a two-dimensional (2D) structure of the aptamer. With reference to a 2D folded structure, a stem of an aptamer can be formed from nucleotide region “b” and nucleotide region “e” of a particular BTX sequence, wherein regions “b” and “e” in the aptamer are based paired. In a variant aptamer, the sequence can be changed to replace a “C” in region “b” and a “G” in region “e,” which are base-paired in the stem, with corresponding nucleotides that maintain the base pairings. For example, contemplated replacements are C→A, T, or G in region “b,” and G→T, A, or C in region “e,” respectively. As such, if the stem is completely replaced with alternate base pairs, the stem structure could still be formed, but the nucleotide sequence of an aptamer variant could have 0% identity to the regions “b” and “e” of the aptamer sequence. In other embodiments, for regions of a variant aptamer sequence that correspond to based-paired regions, such as stem regions, the variant aptamer can have, for example, 0% identity, at least 10% identity, at least 20% identity, at least 30% identity, at least 40% identity, at least 50% identity, at least 60% identity, at least 70% identity, at least 80% identity, at least 90% identity, or 100% identity to the original aptamer sequence in those regions.

Variations in the stem region can also include variations that either lengthen or shorten a stem region. These variations can be reflected by addition of nucleotides to regions of a BTX sequence that form a stem, or deletion of nucleotides to regions of a BTX sequence that form a stem. Preferably, if the variant is defined by deletions to the stem, those deletions do not disrupt the ability of the aptamer to form a stem. Longer stems may permit more deletions, while shorter stems may permit less. In embodiments, the aptamer has variations than result in the loss of 2 or less base pairs, or the loss of only one base pair. In other embodiments, the stem can be lengthened by addition/insertion of nucleotides into stem regions of a BTX sequence, wherein such nucleotide insertions result in 1, 2, 3, 4 or 5 additional base pairs in the stem region. Such additions may increase the stability of a stem and the tertiary structure of the aptamer.

According to some example embodiments, a BTX-binding aptamer can also have one or more nucleotide variations in regions of the aptamer that are not predicted to be based-paired based on 2D modeling, such as loops extending from a stem region, unpaired nucleotide stretches between stems, and unpaired regions at 5′ and 3′ ends of the aptamer sequence. Some or all of these regions may have nucleotides that coordinate with the antigen that the aptamer binds to, and therefore there may be less variability of nucleotide sequence in these regions as compared to the stems. For example, regions of a BTX sequence that correspond to loops extending from a stem region, unpaired nucleotide stretches between stems. Unpaired regions at 5′ and 3′ ends of the aptamer sequence can have 75% or greater identity, at least 80% identity, at least 85% identity, at least 90% identity, at least 92% identity, at least 95% identity, at least 96% identity, at least 97% identity, at least 98% identity, or at least 99% identity, or 100% identity to the original aptamer sequence in those regions. Other example variants of the BTX-binding aptamers of BTX sequences may be further described with reference to 2D structures.

In other examples, the BTX-binding aptamers provided herein may be designed, generated, and tested using other techniques. For example, solid phase synthesis of oligonucleotides using phosphoramidite-based procedures may be used to synthesize oligonucleotides up to about 120 in length. Using any of the aptamer sequences according to the BTX sequences, a large number of nucleotide variants having a specified identity to the starting aptamer sequences (for example, variant sequences having 75% or greater, 80% or greater, 85% or greater, 90% or greater, 95% or greater, or 98% or greater identity to any one of BTX sequences) can be generated and tested for binding to the desired antigen, such as a BTX antigen. Nucleic acid sequences including the BTX-binding aptamers may also be generated and manipulated according to other molecular biology protocols.

A BTX-binding aptamer may, according to some example embodiments, be described in terms of the binding affinity to the BTX antigen. In some embodiments, the BTX antigen-binding aptamer has an equilibrium constant (Kd) of about 1 pM up to about 10.0 μM; about 1 pM up to about 1.0 μM; about 1 pM up to about 100 nM; about 100 pM up to about 10.0 μM; about 100 pM up to about 1.0 μM; about 100 pM up to about 100 nM; or about 1.0 nM up to about 10.0 μM; about 1.0 nM up to about 1.0 μM; about 1 nM up to about 200 nM; about 1.0 nM up to about 100 nM; about 500 nM up to about 10.0 μM; or about 500 nM up to about 1.0 μM.

Binding compounds that include the BTX-binding aptamers may be used as analytical tools in various assay formats, according to some example embodiments. For example, the aptamer-based BTX antigen-binding compounds can be used in solution-based assays or attached to a support surface for immobilized assays.

The BTX-binding aptamers may also be used in diagnostics. Aptamer-based binding molecules may also be used in two-site binding assays, also known as sandwich assays. Generally, in this technique, a BTX antigen is sandwiched between a capture ligand and a detector ligand, with at least one of the ligands being the aptamer-based binding molecule. In some modes of practice, an aptamer-based binding molecule specific for BTX recognizes the BTX antigen, and another aptamer-based binding molecule specific for BTX that is coupled to a fluorophore is used for binding and detection. The assay can be performed in solution without immobilization of either aptamer-based binding molecule.

Aptamer-based binding molecules specific for BTX can also be immobilized on solid supports such as, for example, beads. Such solid support immobilized aptamers can also be used in sandwich assay formats to capture the BTX antigen.

Aptamer-based binding molecules specific for BTX can also be immobilized on other types of surfaces suitable for diagnostic applications, such as nanoparticles made from polymeric materials, metal nanoparticles, including paramagnetic nanoparticles, gold films, gold particles, silicates, silicon oxides, quantum dots, carbon nanotubes, and carbohydrates. Aptamer-based binding molecules specific for BTX can be used in fluorescent, colorimetric, magnetic resonance imaging, or electrochemical sensor detection methods. As such, according to some example embodiments, aptamers described herein can be used to detect a target molecule in a sample.

The aptamer-based binding molecules specific for BTX may be used in a lateral flow assay (LFA). Aptamer-based LFAs include an antigen-aptamer binding reaction combined with lateral fluid flow through a membrane. LFAs can utilize a sandwich format, where two aptamer probes are used for target immobilization and detection. In another embodiment, the LFA arrangement is the competitive format, in which the native antigen competes with an antigen immobilized on a solid support or an ssDNA strand complementary to the aptamer.

According to some example embodiments, kits may be developed with an aptamer-based binding molecule specific for BTX. Such kits may facilitate methods of detection of BTX. Kit components, including the aptamer-containing binding molecules described herein, detection reagents, and optionally other materials, can be, for example, packaged in separate containers and admixed immediately before use. If desired, such packaging of the components can be presented in a pack or dispenser device, which may contain one or more unit dosage forms containing the composition. The pack may, for example, comprise metal or plastic foil, such as a blister pack. Such packaging of the components separately can also, in certain instances, permit long-term storage without losing the activity of the components. Kits may also include reagents in separate containers. Exemplary containers include test tubes, vials, flasks, bottles, syringes, and the like. In some embodiments, kits can be supplied with instructional materials, such as directions for kit use that are printed on paper or other substrates or may be supplied as an electronically readable medium, such as a floppy disc, mini-CD-ROM, CD-ROM, DVD-ROM, Zip disc, videotape, audio tape, and the like. Alternatively, a user may be directed to an Internet website specified by the manufacturer or distributor of the kit.

According to some example embodiments, the BTX-binding aptamers may be employed in therapeutic components for treating infection in, for example, marine organisms. In particular, conjugates of aptamer-based BTX antigen-binding compounds and one or more therapeutic agents may be used. Of particular use are therapeutic agents, such as anti-parasitic drugs, that can be used to treat BTX infections in, for example, marine organisms and, more particularly, fish in aquaculture. Antiparasitic compounds include pyrethroids, cypermethrin, deltamethrin, and other antiparasitics such as praziquantel, mebendazole, albendazole, ivermectin, and levamisole. Other therapeutic agents might be helpful if the marine organism has a secondary infection. In these cases, other therapeutic agents may be used, including organophosphates, benzoylureas, neonicotinoids, amidines, phenols, imidazoles, chloramine-T, methylene blue, beta-lactams, aminoglycosides, tetracyclines, macrolides, chloramphenicol, sulfonamides, potentiated sulfonamides, nitrofurans, quinolones, and fluoroquinolones.

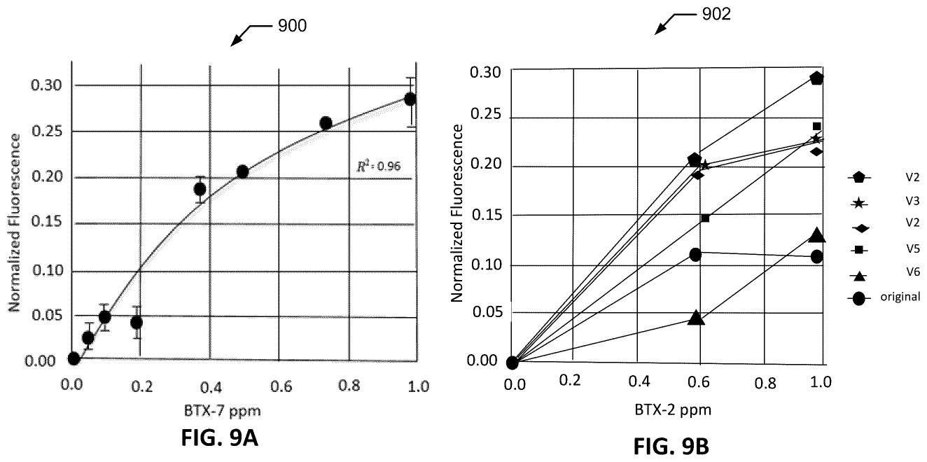

Another example approach for developing BTX-binding aptamers may involve the following. The feasibility of using aptamers in, for example, a FAA was assessed using original, first-generation aptamers having different sequences than those provided above. As shown in the graph 900 of FIG. 9A, a normalized fluorescence calibration curve for BTX-7 using a prototype FAA was generated using the first-generation non-modified aptamers, which showed a 10-fold dynamic range and R² of 0.96. Various modifications to these original aptamer sequences produced five second-generation aptamers (V2, V3, V4, V5, and V6). All aptamers were then tested for sensitivity to BTX-2 and compared (FIG. 9B), showing successful signal enhancement. Four modified second-generation aptamers showed a significantly improved fluorescence response to BTX-2 over the original. For example, the V2 modified aptamer showed an increased detection sensitivity of 300% over the non-modified original aptamer.

Next, a specificity of a FAA fluorescence response was tested with a blank, 8-BTXs at 1 ppm concentration, and 6-small molecules at equimolar concentrations, including domoic acid, another shellfish biotoxin. In all BTX-positive samples, both A- and B-type toxins showed a >1.2× fluorescence response compared with the blank, while the small molecules responded with significantly lower fluorescence validating the assay's specificity for BTXs. Based on this initial data, it appears that FAA in combination with BTX aptamers may be used in developing devices, systems, and methods for detecting BTX.

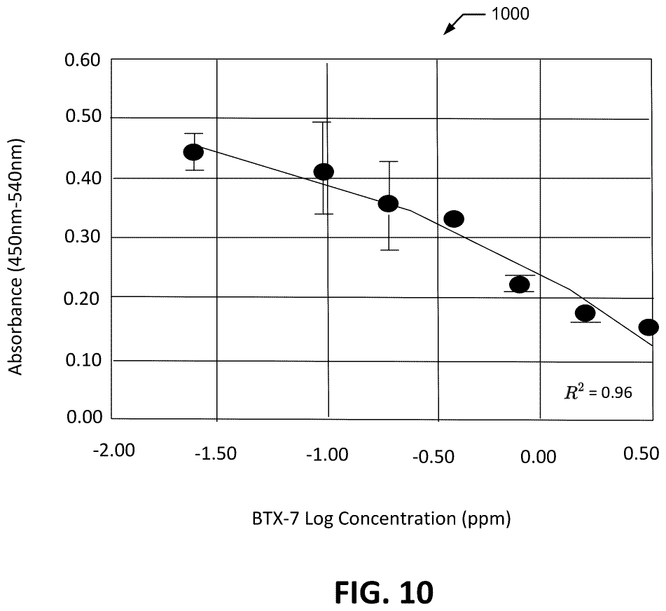

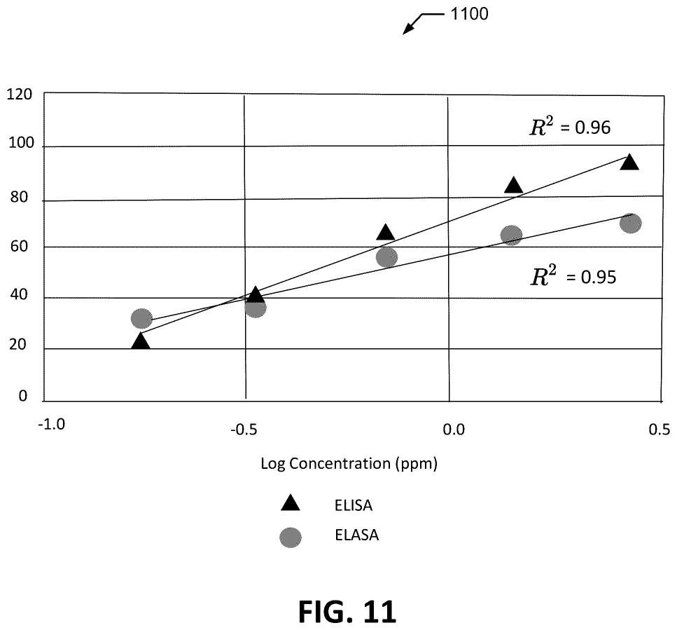

In yet another example, an approach for developing BTX-binding aptamers may involve the following. The feasibility of using ELASA as a BTX detection system was assessed using first-generation aptamers with different sequences than those provided above. As shown in the graph 1000 of FIG. 10, a detection of BTX-7 with a dynamic concentration range from 0.02 ppm to 3.0 ppm by indirect competitive ELASA was observed. A decrease in absorbance corresponded to an increased BTX sample concentration. As shown in the graph 1100 of FIG. 11, regression coefficients for the percent inhibition of the BTX-3 with the MARBIONC ELISA (orange) (type-B toxin; R2=0.96) and BTX-7 with the ELASA (blue) (type-A toxin; R2=0.95) over the same concentration range were almost identical. The ELISA pAbs may be unable to detect type-A toxins, but the ELASA aptamers responded well to both toxin types making the ELASA a more robust assay platform for complete BTX detection.

While first-generation aptamers demonstrated the feasibility of FAA and ELASA devices, it is believed that the higher-affinity BTX aptamer sequences can be leveraged to construct efficient and effective devices, systems, and methods for detecting BTX. While this may include FAA and ELASA, other assays and tests may incorporate the aptamer sequences as well. In turn, this may streamline the harvesting approval process and help minimize economic loss during a red tide bloom. In operation, these methods, devices, and systems may serve as viable alternatives for detecting NSP toxins in shellfish.

As shown below, FAA and ELASA aptamer assays also have more attractive qualities for the end user, like the ease of replenishing aptamers, assay cost, and actual time to complete the assay, directly impacting sample throughput (Table 4).

TABLE 4

| FAA | ELASA | ELISA |
|---|---|---|
| ssDNA Aptamer | ssDNA Aptamer | Goat Polyclonal Ab |
| Easily produced, | Easily produced, | Not quickly produced, |
| no animals required | no animals required | animals required |
| Reproducible | Reproducible | Batch-to-batch variation |
| Low cost to produce kit | Lower cost to produce kit | Moderate cost to produce kit |
| <1 week for aptamer reorder | <1 week for aptamer reorder | 8-10 weeks for antibody |
| | | reorder |
| Time to perform assay <30 | Time to perform assay ≤2 | Time to perform assay = 5 |
| minutes | hours with pre-coated plates | hours, including coating plate |
| | | step |
| Cross-reactive with both A- | Cross-reactive with both A- | Cross-reactive with many B- |
| and B-type BTXs | and B-type BTXs | type BTXs |

From the provided aptamer sequences and work with FAA and ELASA, novel technologies have been developed for detecting a presence of BTX in shellfish and aquatic samples, as described herein. In one example, such samples may include tissue extracts of hard clams, sunray venus clams, and oysters contaminated with a range of BTX concentrations. Additionally, a cross-reactivity of the aforementioned aptamers may be determined for all currently available BTX standards (n=14).

Moving forward, use of FAA and ELASA may continue to be validated through spike-recovery and linearity of dilution testing. Further, such detection methods may be assessed to examine assay stability and ruggedness under various environmental and laboratory conditions. Subsample shellfish extracts may be analyzed using the FAA, ELASA, and MARBIONC ELISA methods and the results correlated to MBA data. In support of screening methods, subsamples may be analyzed and compared and correlated to data from all BTX analyses.

Further detection capabilities and viability may also be assessed. For example, to evaluate the efficacy of the aptamer-based assays as monitoring tools in screening shellfish for BTXs, the linear range (LR), the limit of detection (LOD), the limit of quantitation (LOQ), and the sensitivity of each assay may be determined. The specificity of the assays may be assessed using shellfish extracts spiked with BTX-3 and potential suspected interferences, such as other shellfish toxins. Once the above parameters have been established, shellfish samples with a range of naturally incurred BTX contamination may be assessed to confirm the dynamic range of the assays. Such shellfish samples may include shellfish tissue extracts acquired from three species, hard clam, sunray venus clam, and oyster extracts.

Additionally, as noted above, aptamer cross-reactivity may be evaluated for all available BTX standards. Specifically, the selectivity of the FAA and ELASA to detect both the A- and B-type BTXs may be confirmed by assessing responses to available BTX standards (n=14).

The accuracy of the aptamer-based assays, spike, recovery, and linearity of dilution may continue to improve. The spike and recovery can determine whether toxin detection is affected by a difference between the biological samples (e.g., shellfish matrix) and standard diluent. Linearity of dilution may assess the predictability of the spike on natural recovery for known dilution factors in the desired assay range.

For FAA and ELASA ruggedness (the aptamer-based assays' ability to withstand minor changes in analytical technique, reagents used, or environmental factors (e.g., temperature)), aptamers and reagents may be utilized in aptamer-based assays (e.g., FAA and ELASA). Variations in temperature fluctuations, changes in the incubation duration of samples and aptamers, and development time can be considered. A two-sided t-test (p=≤0.05) may ascertain that the data derived from the above variations is affected by, for example, minor changes in batches/lots, temperature, and incubation time.

In comparison to ELISA and MBA, paired archived tissue extracts previously analyzed by ELISA and the MBA (n=150) can be compared with the FAA and ELASA methods. Tissue extracts may be analyzed with the FAA and ELASA methods following protocols established by QAQC. The accuracy of the aptamer-based assays for BTX detection may be determined by directly comparing results to the current NSSP-approved ELISA and the MBA data. Data may be rigorously correlated and evaluated using linear regression, variance, Spearman rank correlation analyses, and by using R statistics.

Additionally, in comparison and correlation of all BTX analyses to LCMS and support of the developed screening methods, subsamples of shellfish extracts from the methods noted above may be analyzed for toxin identification and quantification using liquid chromatography-mass spectrometry (LCMS). Data generated from the LCMS may be assessed for comparability to FAA and ELASA and current methods (ELISA, MBA) for toxin detection in shellfish. Brevetoxins may be structurally confirmed and quantified with BTX standards using an Agilent LCMS equipped with electrospray ionization.

Finally, data review and assessment of aptamer assays for the product demonstration and manufacturing technology readiness phases can be assessed. Further, training platforms may also be developed in accordance with the methods, devices, and systems provided herein.

According to some example embodiments, a NSP toxin detection technology and rapid lab-based methods, devices, and systems for screening BTXs in shellfish is provided. Unique aptamers have been identified, sequenced, optimized, and then sequenced again for potential BTX detection platforms, e.g., fluorescence activity and enzyme-linked aptasorbent competitive assays. However, other assays and tests may also be used.

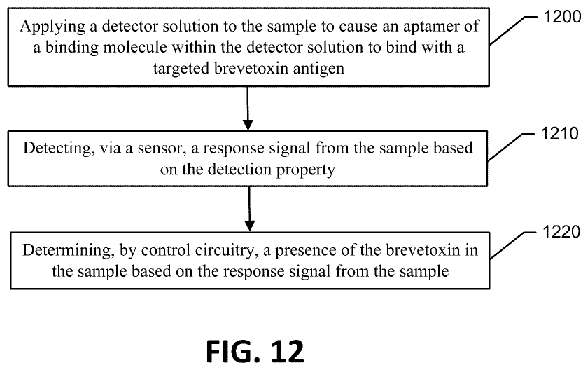

Referring now to the flowchart of FIG. 12, an example method for detecting a brevetoxin is described. At 1200, the method may comprise applying a detector solution (e.g., detector solution 210) to the sample (e.g., sample 220) to cause an aptamer (e.g., BTX-binding aptamer 214) of a binding molecule (e.g., BTX-binding molecule 212) of the detector solution to bind with a targeted brevetoxin antigen (e.g., BTX 222) for the aptamer. In this regard, upon binding of the aptamer to the brevetoxin antigen, a detection property of the detection component (e.g., fluorophore 216 and fluorescent quencher 218) of the binding molecule may be activated. Additionally, at 1210, the method may comprise detecting, via a sensor (e.g., sensor 256), a response signal from the sample based on the detection property, and, at 1220, the method may comprise determining, by control circuitry (e.g., control circuitry 250), a presence of the brevetoxin in the sample based on the response signal from the sample.

The above description is directed to the disclosed methods, devices, and systems and is not intended to limit them. Those of skill in the art will readily appreciate that the teachings found herein may be applied to yet other embodiments within the scope of the present disclosure. Various embodiments are given only by example and are not intended to limit the scope of the present invention. It should be appreciated, moreover, that the various features of the embodiments that have been described may be combined in various ways to produce numerous additional embodiments. Moreover, while various materials, dimensions, shapes, configurations, locations, etc., have been described with disclosed embodiments, others may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of how the various features of the subject matter hereof may be combined. Accordingly, the embodiments are not mutually exclusive combinations of features; instead, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

### SEQUENCE LISTING XML INCORPORATION STATEMENT

The herewith provided Sequence Listing XML is hereby incorporated by reference. The name of the XML file is MOTE68.xml with a date of creation of Nov. 19, 2024 and a size of 45 kilobytes.

What is claimed is:

1. A binding molecule for use in detection of brevetoxins in a sample, the binding molecule comprising:
an aptamer configured to bind to a brevetoxin antigen; and
a detection component having a detection property, the detection component being operably coupled to an aptamer and configured to facilitate detection of a brevetoxin in the sample, via the detection property, upon binding of an aptamer to a brevetoxin antigen.

2. The binding molecule of claim 1, wherein the brevetoxin is a neurotoxic lipid-soluble cyclic polyether compound.

3. The binding molecule of claim 2, wherein the brevetoxin is produced by Karenia brevis.

4. The binding molecule of claim 1, wherein the brevetoxin is a first brevetoxin;
wherein the aptamer is configured to bind with an unsaturated aldehyde tail region of the first brevetoxin and an unsaturated aldehyde tail region of a second brevetoxin, the first brevetoxin and the second brevetoxin having different backbone regions.

5. The binding molecule of claim 1, wherein the aptamer binds to the brevetoxin antigen via a cross-reaction.

6. The binding molecule of claim 1, wherein the aptamer has a nucleic acid sequence with at least one unpaired nucleic acid base and at least one paired nucleic acid base when the aptamer is folded into a double-stranded configuration.

7. The binding molecule of claim 1, wherein the detection component is coupled to the aptamer via a linker component that is a bifunctional crosslinker.

8. The binding molecule of claim 1, wherein the aptamer is configured to, upon binding to the antigen, transition from a first structure to a second structure.

9. The binding molecule of claim 1, wherein detection property of the detection component is activated by the aptamer transitioning from the first structure to the second structure.

10. The binding molecule of claim 1, wherein the detection component comprises a fluorophore.

11. The binding molecule of claim 1, wherein the aptamer comprises a sequence having 60% or greater, 65% or greater, 70% or greater, 75% or greater, 80% or greater, 85% or greater, 90% or greater, 95% or greater, 97% or greater, 98% or greater, 99% or greater, or 100% identity to any one of SEQ ID NOs: 1 to 50.

12. A method of detecting a brevetoxin in a sample, the method comprising:
applying a detector solution to the sample to cause an aptamer of a binding molecule of the detector solution to bind with a targeted brevetoxin antigen, wherein, upon binding of the aptamer to the brevetoxin antigen, a detection property of the detection component of the aptamer is activated;
detecting, via a sensor, a response signal from the sample based on the detection property; and
determining, by control circuitry, a presence of the brevetoxin in the sample based on the response signal from the sample.

13. A test kit for detecting a brevetoxin in a sample, the test kit comprising:
a holder configured to receive the sample; and
a detector solution comprising a binding molecule;
wherein the molecule comprises:
an aptamer configured to bind to the brevetoxin antigen within the sample; and
a detection component having a detection property, the detection component being operably coupled to the aptamer and configured to facilitate detection of a brevetoxin in the sample, via the detection property, upon binding of an aptamer to a brevetoxin antigen.

14. The test kit of claim 10, wherein the test kit is configured to facilitate detection of the brevetoxin via a fluorescent aptamer assay (FAA).

15. The test kit of claim 10, wherein the test kit is configured to facilitate detection of the brevetoxin via an enzyme-linked aptasorbent assay (ELASA).


SEQUENCE LISTING (50 sequences)

SEQ ID NO: 1 - DNA, 48 bp, synthetic construct
  source 1..48: mol_type = other DNA
caccaacggtactaaatgcatgtacgataatgtctctcccgctctgta

SEQ ID NO: 2 - DNA, 49 bp, synthetic construct
  source 1..49: mol_type = other DNA
caccacagaccaaaaatgcatgttacgatagcttaacaccttgccctgg

SEQ ID NO: 3 - DNA, 49 bp, synthetic construct
  source 1..49: mol_type = other DNA
cacgaagggcgcgtaatgcatgttacgattattatccccccttctgtgg

SEQ ID NO: 4 - DNA, 49 bp, synthetic construct
  source 1..49: mol_type = other DNA
cgggaagggcggtaaatgcatgcacgatttgtatctaacctcctcatgg

SEQ ID NO: 5 - DNA, 49 bp, synthetic construct
  source 1..49: mol_type = other DNA
caccaaacgaaaagaatgcatgttacgattattcgcactcgcctctggg

SEQ ID NO: 6 - DNA, 49 bp, synthetic construct
  source 1..49: mol_type = other DNA
caccgaacggaacacatgcatgttacgattaactccacccaactcgtgg

SEQ ID NO: 7 - DNA, 48 bp, synthetic construct
  source 1..48: mol_type = other DNA
caagggcgggtggaaatgcatctacgataatcatctccgcttcccgtg

SEQ ID NO: 8 - DNA, 49 bp, synthetic construct
  source 1..49: mol_type = other DNA
caccgaagaggagatatgcatgttacgatcccttagtatctattgcttg

SEQ ID NO: 9 - DNA, 48 bp, synthetic construct
  source 1..48: mol_type = other DNA
cacaaccgggcgtaaatgcatgctagatatgttcttgtctactcttgg

SEQ ID NO: 10 - DNA, 49 bp, synthetic construct
  source 1..49: mol_type = other DNA
cacgaccagccccaaatgcatgttacgatttgtacactcacgcactttg

SEQ ID NO: 11 - DNA, 49 bp, synthetic construct
  source 1..49: mol_type = other DNA
acacaacgggtttaaatgcatgttacgatacttcaggctattcccttgg

SEQ ID NO: 12 - DNA, 48 bp, synthetic construct
  source 1..48: mol_type = other DNA
cacgaagggcaaattatgcatgcacgatcttaccgttctcaccttccg

SEQ ID NO: 13 - DNA, 49 bp, synthetic construct
  source 1..49: mol_type = other DNA
accacggcagaggtaatgcatgttacgattcaaatcatcccctttggtg

SEQ ID NO: 14 - DNA, 49 bp, synthetic construct
  source 1..49: mol_type = other DNA
cgcaaagggggacatatgcatgctacgataagtagcatcccctctctgg

SEQ ID NO: 15 - DNA, 49 bp, synthetic construct
  source 1..49: mol_type = other DNA
caccccgcagacgaaatgcatgttacgatttctccaactgcactcttgg

SEQ ID NO: 16 - DNA, 48 bp, synthetic construct
  source 1..48: mol_type = other DNA
cacgggaagaacacaatgcatgcacgatatgcagcctttacaccttcg

SEQ ID NO: 17 - DNA, 49 bp, synthetic construct
  source 1..49: mol_type = other DNA
cacaacgggcgacaaatgcatgttacgattgtattgaactcctgacaca

SEQ ID NO: 18 - DNA, 48 bp, synthetic construct
  source 1..48: mol_type = other DNA
cacagcgtaggacctatgcatctacgataatatcctatactgctcttg

SEQ ID NO: 19 - DNA, 49 bp, synthetic construct
  source 1..49: mol_type = other DNA
cacaggggggcatctatgcatgttacgattaagtccacttcacccttgg

SEQ ID NO: 20 - DNA, 49 bp, synthetic construct
  source 1..49: mol_type = other DNA
acccggatggccaaaatgcatgttacgatagtcaatcattccgctcttg

SEQ ID NO: 21 - DNA, 49 bp, synthetic construct
  source 1..49: mol_type = other DNA
accaagggaaggatcatgtatgctacgatactctctcgctaccttctcg

SEQ ID NO: 22 - DNA, 49 bp, synthetic construct
  source 1..49: mol_type = other DNA
cacgggcaggtcatcatgtatgctacgattagctatactacacgccttg

SEQ ID NO: 23 - DNA, 48 bp, synthetic construct
  source 1..48: mol_type = other DNA
cgagaaggaagggaaatgcatgtacgataatcacgcacaccgctcttg

SEQ ID NO: 24 - DNA, 49 bp, synthetic construct
  source 1..49: mol_type = other DNA
caaccggaggataacatgcatgttacgataccccaatgctctccttgtg

SEQ ID NO: 25 - DNA, 49 bp, synthetic construct
  source 1..49: mol_type = other DNA
cgggaagggctaccgatgtatgctacgattcatcactcactcgtcccca

SEQ ID NO: 26 - DNA, 49 bp, synthetic construct
  source 1..49: mol_type = other DNA
ccacgaagcacaaacatgcatgttacgatatgtcaacacgcctttctcg

SEQ ID NO: 27 - DNA, 49 bp, synthetic construct
  source 1..49: mol_type = other DNA
cgacaagagaagagaatgcatgttacgatccctacttttctaaccctgg

SEQ ID NO: 28 - DNA, 49 bp, synthetic construct
  source 1..49: mol_type = other DNA
caccgaagggccagaatgcatgttacgataggacttttatttccgttgg

SEQ ID NO: 29 - DNA, 49 bp, synthetic construct
  source 1..49: mol_type = other DNA
cacgacaggcctaaatgcatggctacgataagtggctttataccccatg

SEQ ID NO: 30 - DNA, 49 bp, synthetic construct
  source 1..49: mol_type = other DNA
cgcaaccaagagacaatgcatgttacgatacacgttcatcctccacttg

SEQ ID NO: 31 - DNA, 49 bp, synthetic construct
  source 1..49: mol_type = other DNA
cacccgagggaagcaatacatgctacgatagaaagtgtgtccttcctgc

SEQ ID NO: 32 - DNA, 49 bp, synthetic construct
  source 1..49: mol_type = other DNA
aacggcagacacgcaatgcatgttacgataaaccgtacattctgccttg

SEQ ID NO: 33 - DNA, 49 bp, synthetic construct
  source 1..49: mol_type = other DNA
accccaacaagagagatgcatgttacgataatcagcctatcatacgcca

SEQ ID NO: 34 - DNA, 48 bp, synthetic construct
  source 1..48: mol_type = other DNA
caccccaagtaccatatgcatgctagataaataggtttccttcttggg

SEQ ID NO: 35 - DNA, 49 bp, synthetic construct
  source 1..49: mol_type = other DNA
cacagcggcgtacccatgcatgttacgatatcaagtgacatcctccgca

SEQ ID NO: 36 - DNA, 48 bp, synthetic construct
  source 1..48: mol_type = other DNA
cacgacatggacattatgcatgctagatatactttcagacctctcttg

SEQ ID NO: 37 - DNA, 48 bp, synthetic construct
  source 1..48: mol_type = other DNA
cacaacaggcagaacatgcatgctagataatcgttgttcacctcttgg

SEQ ID NO: 38 - DNA, 48 bp, synthetic construct
  source 1..48: mol_type = other DNA
caccaacgggtccaaatgcatgctagatactctcacacttcactcgca

SEQ ID NO: 39 - DNA, 49 bp, synthetic construct
  source 1..49: mol_type = other DNA
acccggcaatgggaaatgcatgttacgattattactaccttgctccgta

SEQ ID NO: 40 - DNA, 49 bp, synthetic construct
  source 1..49: mol_type = other DNA
caccacgcaaggggtatgcatgttacgatatactcactaggccatctgg

SEQ ID NO: 41 - DNA, 49 bp, synthetic construct
  source 1..49: mol_type = other DNA
acccaccaggagagcatgcatgttacgatattcttcgcaaccttacggg

SEQ ID NO: 42 - DNA, 48 bp, synthetic construct
  source 1..48: mol_type = other DNA
cgggaggaagaccatatgcatgctagatataatctgaccctccacgtg

SEQ ID NO: 43 - DNA, 49 bp, synthetic construct
  source 1..49: mol_type = other DNA
cacaccagggaactgatgtatgctacgatacttcatccttcctctctgg

SEQ ID NO: 44 - DNA, 49 bp, synthetic construct
  source 1..49: mol_type = other DNA
accgaagagtcaaatatgcatgttacgatacattgctaacccctctttg

SEQ ID NO: 45 - DNA, 49 bp, synthetic construct
  source 1..49: mol_type = other DNA
cacaacaggcgtagtatgcatgttacgattcatcaactgctattcctgg

SEQ ID NO: 46 - DNA, 49 bp, synthetic construct
  source 1..49: mol_type = other DNA
accagaagggcaacaatgaatgctacgattagaattcgcctttgcttgg

SEQ ID NO: 47 - DNA, 48 bp, synthetic construct
  source 1..48: mol_type = other DNA
cacaagcaagcctaaatgcatgctagataatgagtatcctccttgtgg

SEQ ID NO: 48 - DNA, 48 bp, synthetic construct
  source 1..48: mol_type = other DNA
cacagaagggcagatatgcatgctagatcatagtcacttcctatcccg

SEQ ID NO: 49 - DNA, 49 bp, synthetic construct
  source 1..49: mol_type = other DNA
cgggaaggtgaggtaatgtatgctacgatacccccacgcgcttccatcg

SEQ ID NO: 50 - DNA, 48 bp, synthetic construct
  source 1..48: mol_type = other DNA
caacggaagacgacaatgcatgctagatatcattttacgctaccttcg